(19) United States
(12) Patent Application Publication
(10) Pub. No.: US 2005/0250745 A1
(43) Pub. Date: Nov. 10, 2005

(54) Biomarkers for age-related macular degeneration (AMD)

(76) Inventor: Johanna M. Seddon, Boston, MA (US)

Correspondence Address: FISH & RICHARDSON PC, P.O. BOX 1022, MINNEAPOLIS, MN, 55440-1022, US

(21) Appl. No.: 11/066,641

(22) Filed: Feb. 25, 2005

Related U.S. Application Data
(63) Continuation-in-part of application No. PCT/US04/05626, filed on Feb. 25, 2004.
(60) Provisional application No. 60/547,746, filed on Feb. 25, 2004.

(51) Int. Cl. A61K 31/555; A61K 31/60; G01N 33/92; G01N 33/00

(52) U.S. Cl. 514/165; 436/71; 436/86

(57) ABSTRACT

Provided are methods of using levels of markers of systemic inflammation, e.g., CRP, to predict a subject's risk of development or progression of Age-Related Macular Degeneration (AMD), and methods of treating, delaying or preventing the development or progression of AMD.

### CLAIM OF PRIORITY

This application is a continuation-in-part of PCT/US04/05626, filed on Feb. 25, 2004, and claims the benefit of U.S. Patent Application Ser. No. 60/547,746, filed on Feb. 25, 2004, the entire contents of which are hereby incorporated by reference.

### FEDERALLY SPONSORED RESEARCH OR DEVELOPMENT

This invention was made with Government support under Grant No. RO1EY13982, and Contracts Nos. NO1EY02117, and NO1EY02126, awarded by the National Institutes of Health. The Government has certain rights in the invention.

### TECHNICAL FIELD

This invention relates to methods of using biomarkers, including markers of systemic inflammation, as diagnostic and prognostic tools for age-related macular degeneration (AMD), and methods for treating, preventing or delaying the development or progression of AMD.

### BACKGROUND

Age-related macular degeneration (AMD) is a sudden worsening and distortion of central vision that progresses rapidly, typically with a course of only weeks or months. AMD is characterized by abnormalities in the macular area. The central area (or fovea) of the macula contains the highest density of cone photoreceptors in the retina and mediates high-acuity vision. The disease typically has a preclinical, asymptomatic phase, in which extracellular waste material accumulates in the space between the basement membrane (Bruch's membrane) and the epithelial layer, forming yellow-white spots known as drusen. Advanced forms of AMD includes both dry and wet (or “neovascular”) AMD. The dry form of AMD is far more common, but the wet form occurs simultaneously with the dry form in about 15% of cases. Dry AMD is characterized by progressive apoptosis of cells in the epithelial layer, in the overlying photoreceptor cells and in the underlying cells in the choroidal capillary layer. Wet AMD is characterized by choroidal neovascularization with vascular leakage into subretinal spaces.

AMD impairs central vision that is required for reading, driving, face recognition, and fine visual tasks. Neurosensory detachment, retinal hemorrhages, and retinal scarring gradually result in decreased visual function of photoreceptors in the central vision, eventually resulting in legal blindness, with preservation of peripheral vision. AMD is the most common cause of blindness among the elderly. Subjects with a family history of AMD and those who smoke have a higher risk than non-smokers and those with no family history, however, subjects who have favorable risk profiles also develop the disease.

Current therapeutic efforts and clinical trials are primarily aimed at halting the growth of the neovascular membrane in wet AMD, e.g., using angiogenesis inhibitors, laser photocoagulation, and/or photodynamic therapy. Antioxidants can retard the progression of the disease. Despite advances in treatment, AMD is still the most common cause of visual impairment in the developed world.

### SUMMARY

The present invention is based, in part, on the discovery of a relationship between certain AMD biomarkers, e.g., lipid biomarkers and markers of systemic inflammation, e.g., C-Reactive Protein (CRP) and Interleukin-6 (IL-6), and risk of developing and progression of Age-Related Macular Degeneration (AMD). Thus, in one aspect, the invention provides new diagnostic methods that determine the levels of AMD biomarkers, including lipid biomarkers and markers of systemic inflammation, which information can be used to predict a subject's risk of developing AMD and/or progressing to more advanced AMD. These methods can be used, e.g., to predict risk of development or progression of age-related maculopathy, to select subjects for a clinical trial, and/or to determine the likelihood that specific subjects will benefit from certain treatments (e.g., the administration of anti-inflammatory agents) for non-neovascular as well as neovascular AMD. The invention also provides methods for treating, preventing, and/or delaying the development or progression of AMD.

This invention describes new diagnostic and prognostic methods that determine and utilize the levels of AMD biomarkers, including markers of systemic inflammation, e.g. to determine the magnitude of systemic inflammation. These new methods broadly include the prediction of risk of development and/or progression of AMD; and the determination of the likelihood that certain subjects will benefit from the use of certain treatments designed to prevent development or progression of AMD and/or treat AMD. These new methods are based in part upon the discoveries described herein.

As described herein, elevated levels of AMD biomarkers, e.g., markers of systemic inflammation, e.g., CRP and IL-6, Tumor Necrosis Factor alpha-Receptor II (TNF-R2), Intercellular Adhesion Molecule (ICAM), and/or Vascular Cell Adhesion Molecule (VCAM); lipid biomarkers, e.g., Apolipoprotein B (ApoB) or Lipoprotein (a) (LP(a)); and homocysteine, are predictive of development and progression of AMD. Elevated levels of these AMD biomarkers in otherwise healthy subjects, regardless of whether they have ever smoked, are predictive of development and progression of AMD.

In addition, the likelihood that a specific subject will benefit to a greater or a lesser extent from the use of certain therapeutic agents (e.g., anti-inflammatory agents) for reducing the risk of development or progression of AMD can be determined by evaluating levels of AMD biomarkers, e.g., markers of systemic inflammation, e.g., levels of CRP, IL-6, Tumor Necrosis Factor alpha-Receptor II (TNF-R2), Intercellular Adhesion Molecule (ICAM), and/or Vascular Cell Adhesion Molecule (VCAM), in the subject, and comparing the levels to a reference for the marker.

The predictive value of the biomarkers described herein for risk of development or progression of AMD is independent of other predictors. In fact, risk prediction based on levels of these markers is additive with some other risk factors, and does not simply duplicate the information derivable from evaluating those other factors (e.g., smoking, obesity, body mass index, fat intake, antioxidant vitamin and mineral intake, age, sex, family history of AMD, and physical exercise levels, inter alia).

In one aspect, the invention provides methods for characterizing a subject's risk of development or progression of AMD. The methods include obtaining a level of a biomarker, e.g., a biomarker as described herein, e.g., a marker of systemic inflammation in the subject or a lipid biomarker, e.g., levels of CRP, IL-6, ApoB, TNF-R2, HCY, ICAM or VCAM. The level of the marker is compared to a reference, and the subject's risk of development or progression of AMD is characterized based upon the level of the marker in comparison to the reference. In some embodiments, the level of the marker is higher than the reference, and it indicates that the subject has an increased risk of development or progression of AMD, e.g., increased relative to a control or other reference (including a previous level obtained from the same subject). In some embodiments, the level of the marker is lower than the reference, and it indicates that the subject has a reduced risk of development or progression of AMD, e.g., reduced relative to a control or other reference (including a previous level obtained from the same subject). In some embodiments, the method also includes selecting a treatment option based on the level of an AMD biomarker, e.g., a lipid biomarker or a marker of systemic inflammation; in one embodiment, a level of a marker of systemic inflammation is high, and a treatment option including administering an anti-inflammatory agent is selected. As used herein, progression of AMD refers to an increase in severity of the disease, e.g., an objective worsening in one or more parameters or symptoms associated with the disease, e.g., a progression from group 1 or 2 as described in Example 1, to group 3 or 4, or a progression from group 1, 2, or 3 to group 4. In some embodiments, the subject is re-evaluated, e.g., the level of an AMD biomarker is obtained again after or during administration of a treatment (e.g., after administration of one or more doses of an anti-inflammatory agent), and the level is compared to a reference, e.g., the level previously obtained, to evaluate the efficacy of the treatment. In some embodiments, after administration of the treatment, the level of an AMD biomarker, e.g., a marker of systemic inflammation, is reduced as compared to a previously-obtained level, and it indicates that the subject's risk or development or progression of AMD is reduced.

According to yet another aspect of the invention, methods are provided that use a level of an AMD biomarker, e.g., levels of CRP, IL-6, ApoB, TNF-R2, HCY, ICAM or VCAM, together with one or more risk factors, e.g., as described herein, to characterize a subject's risk profile of development and/or progression of AMD. The methods include obtaining a level of a biomarker, e.g., a marker of systemic inflammation in the subject. The level of the biomarker is compared to a reference to establish a first risk value. At least one risk factor is also evaluated. The presence or level of the risk factor in the subject is compared to a second reference to establish a second risk value. The subject's risk profile for development or progression of AMD is characterized based upon the combination of the first risk value and the second risk value, wherein the combination of the first risk value and second risk value establishes a combined risk value, which is typically different from the first and second risk values. In some embodiments, the combined risk value is greater than either of the first and second risk values, e.g., the first and second risk values are additive.

In another aspect of the invention, methods are provided for evaluating the likelihood that a subject will benefit from treatment with an agent, e.g., an anti-inflammatory agent, to reduce the risk of development or progression of AMD. The methods include obtaining a level of an AMD biomarker, e.g., a marker of systemic inflammation in a subject. This level then is compared to a reference, wherein the level of the AMD biomarker in comparison to the reference is indicative of the likelihood that the subject will benefit from treatment with the agent. The subject then can be characterized in terms of the net benefit likely to be obtained by treatment with the agent.

In a further aspect, the invention provides methods for determining whether a treatment or prevention for reducing risk of development of age-related macular degeneration (AMD) or progression to advanced AMD is having an effect in a subject. The methods include obtaining a first level of an AMD biomarker, e.g., a lipid biomarker or a marker of systemic inflammation in a subject; administering a selected treatment or prevention for AMD to the subject, e.g., as described herein; obtaining a second level of the AMD biomarker in the subject; and comparing the first level of the AMD biomarker to the second level of the AMD biomarker. A difference, or lack of difference, in the second level as compared to the first level indicates whether the treatment or prevention is effective or not effective. For example, if the second level is lower than the first level, then the treatment or prevention is likely to be effective. If there is no difference, or if the second level is higher than the first level, that the treatment or prevention is not effective, or has not yet become effective. The methods can include re-testing the subject a plurality of times, e.g., to determine whether the treatment is effective over time. In addition, once a treatment has been stopped, the subject can be tested one or more additional times to determine if their risk has changed.

In some embodiments, the subject is apparently healthy, e.g., has no or few overt clinical signs of AMD (e.g., is in the first maculopathy group as described in Example 1); has minimal or early AMD (e.g., is in the second maculopathy group); has intermediate AMD (e.g., is in the third maculopathy group); or has advanced AMD (e.g., is in the fourth maculopathy group). In some embodiments, the subject is a non-smoker, e.g., has never smoked, or is a smoker, e.g., a current or past smoker. A non-smoker is a subject who, at the time of the evaluation, has never smoked, or has smoked less than a minimal number, e.g., 100 cigarettes in a lifetime, or less than a minimal duration, e.g., less than six months. Smokers include subjects who currently smoke, as well as subjects who have smoked at some time in the past but presently no longer smoke, or smoked more than a minimal number, e.g., 100 cigarettes in a lifetime, or more than a minimal duration, e.g., more than six months. In some embodiments, the subject has no risk factors as described herein. In some embodiments, the subject has one or more risk factors as described herein.

In some embodiments, characterizing a subject's risk of development or progression of AMD includes characterizing the subject's risk of developing advanced AMD. In some embodiments, characterizing the subject's risk of future development or progression of AMD includes characterizing the subject's risk of developing neovascular AMD.

The reference can be a single value, multiple values, a single range or multiple ranges. In some embodiments, the reference is a median value. In some embodiments, the reference is a plurality of marker level ranges, e.g., ranges associated with low, medium, and high risk categories, and the comparing step comprises determining in which marker level range the subject's level falls. Exemplary references are shown in Tables A and B.

In some embodiments, levels of multiple AMD biomarkers, e.g., markers of systemic inflammation are obtained concurrently. In some embodiments, levels of one or more lipid biomarkers are obtained in place of or in addition to a marker of systemic inflammation. In some embodiments, the lipid biomarkers are apolipoprotein B (ApoB) or lipoprotein (a) (Lp(a)).

One of skill in the art will appreciate that the reference value selected will typically depend on the particular marker selected and even upon the characteristics of the patient population in which the subject lies, described in greater detail below.

As mentioned above, the methods described herein can be adapted to determine which subjects are most likely to benefit from treatment with an agent for reducing the risk in the development or progression of AMD. The methods can also be used to select candidate subjects and/or populations for clinical trials and for treatment with candidate drugs, by identifying, for example, subjects most likely to benefit from a new treatment or from a known treatment with a high risk profile of adverse side effects. Thus, the methods described herein can provide information for evaluating the likely net benefit of certain treatments for candidate subjects.

The invention also includes kits including a package including one or more assays for an AMD biomarker as described herein, e.g., a lipid biomarker (e.g., ApoB) and/or a marker of systemic inflammation (e.g., CRP, IL-6), and instructions for use in a method described herein, and optionally related materials such as marker level or range information for correlating the level of the marker as determined by the assay with a risk of development or progression of AMD. Such information can be in any useful form, e.g., charts, e.g., numeric or color charts. In some embodiments, the instructions include information for determining the subject's risk of development or progression of AMD, by correlating the level of the marker determined by the assay and one or more risk factors with a risk of development or progression of AMD. In some embodiments, the kit includes assays for two, three, four or more AMD biomarkers. For example, the kit can include a microarray or a microfluidic device that can be used to determine the levels of two, three, four, or more AMD biomarkers, e.g., substantially simultaneously.

The invention also involves methods for treating subjects with anti-inflammatory therapies, to treat, prevent, and/or delay the development or progression of AMD. In some embodiments, a non-aspirin anti-inflammatory agent is administered to a subject who is known to have (i.e., has been determined to have, e.g., by a method described herein) an above-normal level of a marker of systemic inflammation, but who is otherwise free of symptoms calling for an anti-inflammatory agent. The anti-inflammatory agent is administered in an amount effective to treat, prevent, and/or delay the development or progression of AMD. In some embodiments, the anti-inflammatory agent is administered in an amount effective to reduce the subject's levels of one or more selected AMD biomarkers, e.g., CRP and/or IL-6, e.g., to below a preselected value, e.g., a reference value (e.g., as described herein) that is associated with a lower risk category.

In some embodiments, the subjects are apparently healthy subjects as described herein.

In an additional aspect, the invention provides packages including an anti-inflammatory agent, e.g., in a pharmaceutical composition, and instructions for administering the anti-inflammatory agent to a subject in order to treat, prevent, and/or delay the development or progression of AMD. In some embodiments, the anti-inflammatory agent is in a therapeutic composition also including a pharmacologically acceptable carrier. In some embodiments, the anti-inflammatory agent is in a form suitable for local delivery to the macular area.

As used herein, “age-related macular degeneration” or “AMD” includes both early, intermediate, and advanced AMD. “Advanced AMD” includes both dry AMD and wet AMD (wet AMD is also referred to as neovascular AMD). Subjects with advanced AMD are those who can be categorized in group 4 as described in Example 1, or in groups 4 and/or 5 as described in Example 4. An “AMD biomarker” is a marker associated with development of AMD, or progression to advanced AMD, in a subject, as described herein.

As used herein, a “subject” is a mammal, e.g., human, canine, feline, ovine, primate, equine, porcine, caprine, camelid, avian, bovine, and murine organisms. Typically, the subject is a human. As used herein, “apparently healthy” means that a subject does not have clinical signs of AMD, e.g., is in the first maculopathy group as described herein, and is essentially free of current need for anti-inflammatory treatment, such as free of symptoms of rheumatoid arthritis, chronic back pain, autoimmune diseases (e.g., amyotrophic lateral sclerosis, multiple sclerosis, type I diabetes, graft-versus-host disease, rheumatoid arthritis, inflammatory bowel disease, uveitis, and thyroiditis), and the like. In other words, such subjects, if examined by a medical professional, would be characterized as healthy and generally free of symptoms of acute disease.

As used herein, a “risk factor” means a risk factor that is known to be associated with an increased risk of development or progression of AMD.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a bar graph illustrating the adjusted odds ratios for prevalence of maculopathy according to CRP level and smoking status as shown in Table 5.

FIG. 2 is a bar graph illustrating progression rate (%) according to level of CRP. Total number of subjects in respective categories: CRP<0.5 (N=30), 0.5-<5.0 (N=144), 5.0-<10.0 (N=34), 10+ (N=43)

FIG. 3 is a bar graph illustrating progression rate (%) according to level of IL-6. Total number of subjects in respective categories: IL-6<2.0 (N=81), 2-3.9 (N=91), 4-5.9 (N=29), 6+ (N=48)

### DETAILED DESCRIPTION

As described herein, it has been discovered that elevated levels of CRP and IL-6, markers of systemic inflammation, are predictive of AMD. This association is present for both smokers and non-smokers. It has been further discovered that elevated levels of CRP predict risk of AMD, and elevated levels of CRP and IL-6 predict risk of progression of AMD, independent of other risk factors, including dietary fat and higher body mass index. Thus, information about this biomarker does not duplicate, and is likely to be independent of, the information provided by other known or presumed risk factors. Even when numerous other known risk factors are controlled for (see Examples, below), the inflammatory markers improve prediction. Furthermore, using an AMD biomarker as an assay for AMD risk can provide a simpler method of evaluating a subject's risk that does not require information about a subject's personal medical history.

The results described herein implicate a number of biomarkers, e.g., systemic inflammatory markers such as CRP and IL-6, as markers for the development and progression of AMD. Higher CRP and IL-6 values were found to be significantly related to AMD, independent of established risk factors including smoking and obesity. Among smokers and non-smokers, higher baseline levels of CRP and/or IL-6 were associated with an increased risk of AMD. These results shed light on the mechanisms and pathogenesis of AMD development and prognosis. Moreover, CRP and IL-6 levels add clinically relevant predictive information concerning risk of AMD in addition to known risk factors. Anti-inflammatory agents thus may have a role in preventing and/or treating AMD, and biomarkers such as CRP provide a method of identifying people for whom these agents would be more or less effective.

As described herein, elevated levels of markers of systemic inflammation (e.g., CRP, IL-6, TNF-R2, HCY, ICAM and VCAM) are associated with development and/or progression of AMD. After adjustment for age, sex, and other variables including smoking and body mass index, CRP and IL-6 levels were significantly higher among subjects with intermediate and advanced stages of AMD compared with controls (e.g., subjects in the first and/or second maculopathy groups, as described herein). The magnitude of effect ranged from an odds ratio (OR) of 1.65 to 2.16 for the highest levels of CRP. Similar but less significant results were observed for other markers of systemic inflammation, e.g., TNF-R2, ICAM and VCAM, as well as lipid biomarkers, apolipoprotein B (ApoB) and lipoprotein a (LP(a)), and homocysteine (HCY). Anti-inflammatory agents thus may have a role in slowing the progression to advanced AMD, and these biomarkers provide a method of identifying people for whom these agents would be more or less effective.

The odds ratio refers to the odds of getting a disease, if a selected factor is present. For example, an OR of 2 refers to a two-fold increase in risk of getting the disease over a reference. Risk of AMD was lowest among those with low CRP and IL-6 values who never smoked. In contrast, risk tended to be highest among smokers who also had higher levels of CRP and/or IL-6. Even among those who never smoked, the risk of AMD was increased two-fold among those with the highest category of CRP and/or IL-6, compared with the lowest level of CRP and/or IL-6 as the referent category.

The findings described herein demonstrate that inflammation is associated with the pathogenesis of AMD. Several mechanisms that could lead to inflammatory responses may be involved including oxidative stress caused by risk factors for AMD, such as smoking (Seddon et al., JAMA 1996; 276:1141-1146; Smith et al., Ophthalmology 2001; 108: 697-704), insufficient antioxidants in the diet (Seddon et al., JAMA 1994; 272:1413-1420; Cho et al., Am J Clin Nutr 2001; 73: 209-218), dietary fat (Seddon et al., Arch Ophthalmol 2001; 119:1191-1199; Cho et al., Am J Clin Nutr 2001; 73: 209-218), or obesity (Seddon et al., Arch Ophthalmol 2003; 121:785-792). Smoking is one of the most consistent risk factors for AMD, yet many subjects who have never smoked develop AMD. As described herein, higher CRP and/or IL-6 values were strongly associated with increased risk of AMD among smokers as well as among those who never smoked, independent of the other risk factors in the model. Therefore, it is likely that factors other than smoking in these subjects create an adverse milieu or damage the RPE-retina-choroidal complex in some way, which in turn leads to an inflammatory stimulus and increased CRP and/or IL-6 values.

The study described herein evaluated systemic biomarkers, including markers of systemic inflammation, in a large and well-characterized population of subjects with and without maculopathy from two geographical areas in the United States. Standardized collection of risk factor information including direct measurements of blood pressure and body mass index, as well as classification of maculopathy by means of standardized ophthalmological examinations and fundus photography, was employed. Misclassification was unlikely because CRP and IL-6 values were quantified using objective laboratory methods without knowledge of the subjects' maculopathy status, and AMD grade was assigned without knowledge of CRP or IL-6 status.

Residual confounding is a concern in many epidemiological studies. Known AMD risk factors and those associated with AMD in this study cohort were controlled for. For example, obesity and cigarette smoking are related to AMD, and are also related to increased levels of CRP and other systemic inflammatory markers (Visser et al., JAMA 1999; 282:2131-2135). As described herein, increased CRP and IL-6 levels were significantly and independently related to AMD in this study after adjustment for these confounding factors. Although some unmeasured and therefore uncontrolled factors might still be confounding this relationship, they would have to be both highly associated with CRP or IL-6, and a strong risk factor for AMD to explain these results.

The study population consisted of subjects with a range of maculopathy and some subjects without AMD who participated in a randomized trial of nutritional supplements. Results were not altered after adjustment for assignment to antioxidants within the randomized trial. Controls were more likely to be female, non-smokers, and with more education. However, these analyses were adjusted statistically for these differences, and previous case-control analyses of the entire Age-Related Eye Disease Study (AREDS) cohort, as well as this subset at two centers, demonstrated an association with known risk factors for AMD similar to other study populations. Although the study population was a selected population, these cases likely represent the typical subject with AMD. In addition, the study population overall was comparable to the general population in this age range in terms of smoking status and prevalence of obesity.

Measures of CRP were taken from single fasting blood specimens that were stored in a repository at −140° C. until analyzed. These are standardized methods that are in use in several large-scale epidemiologic studies throughout the country (Ridker et al., JAMA 2001; 285:2481-2485; Ridker, Circulation 2003; 107:363-369). The medians and ranges of CRP in the various quartiles in this study are similar to other published studies of CRP and cardiovascular diseases (Id.).

Biomarkers Associated with Development of AMD, or Progression to Advanced AMD

In some embodiments, the methods described herein include determining a level of an AMD biomarker, e.g., a marker associated with development of AMD, or progression to advanced AMD, e.g., a marker of systemic inflammation, in a subject. Markers of systemic inflammation are known to those in the art. In some embodiments, the markers of systemic inflammation can be C-reactive protein, cytokines, tissue necrosis factor alpha receptor-II (TNF-R2), and cellular adhesion molecules. Cytokines are known to those in the art and include human interleukins 1-17 (IL-1 through IL-17); in some embodiments, the marker is interleukin-6 (IL-6). Cellular adhesion molecules are known to those in the art and include integrins, intracellular adhesion molecules (e.g., ICAM-1, ICAM-3), B-lymphocyte cell adhesion molecule (BL-CAM), lymphocyte function-associated adhesion molecules (e.g., LFA-2), vascular cell adhesion molecules (e.g., VCAM-1), neural cell adhesion molecule (NCAM), platelet endothelial cell adhesion molecule (PECAM), and soluble intercellular adhesion molecule (sICAM-1). In some embodiments, the marker is an intracellular adhesion molecule (ICAM) or a vascular adhesion molecule (VCAM). In some embodiments, the AMD biomarker is homocysteine (HCY), an amino acid produced as a normal byproduct of the breakdown of methionine. In some embodiments, the AMD biomarker is a lipid biomarker, e.g., apolipoprotein B (ApoB) or lipoprotein A (LP(a)).

In some embodiments, an AMD biomarker is a component of blood, e.g., is present in the blood of a subject. AMD biomarkers can be, e.g., polypeptides, peptides, lipoproteins, lipids, and amino acids (e.g., homocysteine). In some embodiments, an AMD biomarker is a marker associated with increased risk of cardiovascular disease.

Levels of AMD Biomarkers

A level of an AMD biomarker in a subject can be obtained by any art recognized method. Typically, the level is obtained by measuring the level of the marker in a sample including a body fluid, for example, blood, lymph, saliva, urine, and the like. The level can be determined by immunoassays, e.g., enzyme-linked immunoassays (EIA) or enzyme-linked immunosorbent assays (ELISA); particle agglutination or flocculation tests (e.g., rapid latex agglutination); laser and rate nephelometry; turbidometry; or other known techniques for determining the presence and/or quantity of the marker.

The methods can include obtaining a level of an AMD biomarker in a subject by sending one or more samples of the subject's body fluid to a laboratory, e.g., a commercial laboratory, for measurement of levels. In some embodiments, the methods include measuring the level of the biomarker in a body fluid from a subject, and providing information regarding the level of the biomarker, e.g., to the subject or a caregiver, e.g., a clinical entity such as a physician, nurse, hospital, clinical practice, or third-party payor, e.g., an insurance company.

Reference Values

In some embodiments, the methods described herein also include comparing the level of an AMD biomarker for the subject with a reference. The reference can take a variety of forms. It can be a single cut-off (threshold) value, such as a median or mean. It can be established based upon comparative groups, such as where the risk in one defined group is higher than, e.g., double, the risk in another defined group. It can be a range, for example, where the tested population is divided equally (or unequally) into groups, such as a low-risk group, a medium-risk group, and a high-risk group, or into quintiles (fifths), quartiles (quarters), or tertiles (thirds), the lowest group being subjects with the lowest risk and the highest being subjects with the highest risk.

The reference can depend upon the particular population selected. For example, an apparently healthy, nonsmoker population (no detectable disease and no prior history of AMD) can have a different “normal” range of markers of systemic inflammation than a smoking population, or a population the members of which have some stage of AMD. Accordingly, the reference selected may take into account the category in which a subject falls. Appropriate ranges and categories can be selected with no more than routine experimentation by those of ordinary skill in the art. In some embodiments, the reference is a predetermined value, e.g., as shown in Table A or B. These values are approximate.

TABLE A
Categories of Risk of Developing AMD

| Biomarker (median) | Lowest Risk | Average Risk | Highest Risk |
|---|---|---|---|
| CRP (2.7 mg/L) | <1.7 mg/L | 1.7 mg/L-4.5 mg/L | >4.5 mg/L |

TABLE B
Categories of Risk of Progression to Advanced AMD

| Biomarker (median) | Lowest Risk | | Average Risk | | Highest Risk | |
|---|---|---|---|---|---|---|
| CRP (2.5 mg/L) | <0.5 | mg/L | 0.5-10 | mg/L | >10.0 | mg/L |
| IL-6 (2.72 pg/ml) | <2 | pg/ml | 2 pg/ml-6 pg/ml | | >6 | pg/ml |
| ApoB (100.2 mg/dl) | <75 | mg/dl | 75-100 | mg/dl | >100 | mg/dl |
| VCAM (977.9 ng/ml) | <700 | ng/ml | 700-1000 | ng/ml | >1000 | ng/ml |
| TNF-R2 | <2150 | pg/ml | 2150-4000 | pg/ml | >4000 | pg/ml |
| (3215.5 pg/ml) | | | | | | |

In some embodiments, the body fluid is blood and the marker is C-reactive protein and/or IL-6. For C-reactive protein, one important reference value, e.g., for a population of apparently healthy nonsmokers, is 2.7 mg/liter (the median for the subject population). Values above this reference indicate an increased risk of developing AMD. For IL-6, an important reference value is 3 pg/ml. Values above this reference indicate an increased risk of developing AMD.

Commercially available assays and reagents can be used for measuring levels of C-reactive protein. Commercial sources for these reagents and assays include, e.g., CalBiochem (San Diego, Calif.), Roche Diagnostics Corp. (Indianapolis, Ind.), Ortho-Clinical Diagnostics, Inc. (Rochester, N.Y.), and Johnson & Johnson Clinical Diagnostics, Inc. (Rochester, N.Y.), among many others. The U.S. Food and Drug Administration (FDA) regulates such diagnostic tests, and CRP immunoreactive tests are categorized by the FDA under section 866.5270; assays, reagents, and devices listed thereunder can be used in the methods described herein to obtain the CRP level of a subject. A number of assays for levels of inflammatory cytokines, lipid markers, homocysteine, and cellular adhesion molecules are known in the art. For example, commercial sources of assays for inflammatory cytokine and cellular adhesion molecule, include, but are not limited to, R&D Systems (Minneapolis, Minn.), Genzyme (Cambridge, Mass.), and Immunotech (Westbrook, Me.).

Also provided are novel kits or assays that are specific for, and have appropriate sensitivity with respect to, biomarker references selected on the basis of the present invention. In some embodiments, therefore, the kits or assays would differ from those presently commercially available, by including, for example, different cut-offs, and/or different sensitivities at particular cut-offs as well as instructions or other printed material for characterizing risk of developing AMD or progressing to advanced AMD, based upon the outcome of the assay.

Risk Factors for AMD

A number of risk factors are known to be associated with an increased risk of development or progression of AMD. In some embodiments, the risk factor is family history of AMD, increasing age (e.g., above 60, 70, 80 or 90 years of age), sex (women have a higher risk), smoking history, obesity, e.g., high body mass index (BMI, e.g., a BMI of >25), high waist circumference, high waist-hip ratio, weight change since age 20, dietary fat intake, high linoleic acid intake, and/or elevated cholesterol levels, e.g., as described in Seddon et al., Arch Ophthalmol. 2003 June; 121(6):785-92, and Seddon et al., Arch Ophthalmol. 2003 December; 121(12):1728-37. Erratum in: Arch Ophthalmol. 2004 March; 122(3):426.

In some embodiments, the risk factor is the presence or absence of a genetic marker, e.g., as described in Weeks et al., Am J Hum Genet 75:174-189, 2004. For example, in some embodiments the genetic marker is a four-marker single-nucleotide polymorphism (SNP) haplotype in the locus spanning the gene ALOX5AP encoding 5-lipoxygenase activating protein (FLAP) (see Helgadottir et al., Nat Genet. 2004 March; 36(3):233-9. Epub 2004 Feb. 08). In some embodiments, the genetic marker is on chromosome 1q (236-140 cM in the Marshfield genetic map), 2p (10 cM), 5p (40-50 cM), 9q (111 cM), and/or 22q (25 cM) (see Abecasis et al., Am. J. Hum. Genet. 74:482-494, 2004). See, e.g., Seddon et al., Am J Hum Genet. 2003 October; 73(4):780-90. Epub 2003 Aug. 22. Other genes include Fibulin 5 (Stone et al., NEJM 351:346-53, 2004), PON1 (Ikeda et al., Am J Ophthalmol 132:191-5, 2001), CST3 gene (on chromosome 20p, Zurdel et al., Br J Ophthalmol 86:214-9, 2002)), SOD2 (on chromosome 6q, Kimura, Am J Ophthalmol 130:769-73, 2000), angiotensin-converting enzyme (ACE; on chromosome 17q, Hamdi et al., Biochem Biophys Res Commun 295:668-72, 2002), and CX3CR1, a chemokine receptor gene (on chromosome 3p, Tuo et al., FASEB J 18:1297-9, 2004), and apolipoprotein E (APOE), ABCA4 (ABCR), and HEMICENTIN-1 (Fibulin 6), located on chromosomes 19q, 1p, and 1q respectively.

In some embodiments, a risk factor is a level of an AMD biomarker as described herein, e.g., a marker of systemic inflammation or a lipid biomarker.

Methods for Predicting and Evaluating the Efficacy of a Treatment

Also provided herein are methods for evaluating the likelihood that a subject will benefit from treatment with an anti-inflammatory agent for reducing risk of development or progression of AMD. The method includes determining the level of a biomarker, e.g., a marker of a systemic inflammation (e.g., CRP and/or IL-6) in the subject; if the level of the marker is high, then the subject is likely to benefit from the administration of an anti-inflammatory agent. In some embodiments, the method can further include administering an anti-inflammatory agent to the subject.

The methods described herein can also be used to evaluate the efficacy of a treatment for reducing the risk of development or progression of AMD. For example, the method can include determining the level of one or more AMD biomarkers, e.g., markers of systemic inflammation (e.g., CRP and/or IL-6) before, concurrently with, and/or after the administration of the treatment. In some embodiments, the subject receives multiple treatments, e.g., a treatment is administered in multiple doses, e.g., one or more doses per day for one or more days, weeks, months, or years, and the level of an AMD biomarker, e.g., a marker of systemic inflammation (e.g., CRP and/or IL-6) is determined, e.g., before any treatment, and after one or more treatments. In some embodiments, the treatment is the administration of an anti-inflammatory agent, e.g., as described herein. In some embodiments, the methods described herein are performed as part of a clinical trial of a treatment to reduce the risk of the development or progression of AMD.

These methods have important implications for subject treatment and also for clinical development of new therapeutics. Physicians typically select therapeutic regimens for subject treatment based upon the expected net benefit to the subject. The net benefit is derived from the risk to benefit ratio. The present methods permit selection of subjects who are more likely to benefit by intervention, thereby aiding the physician in selecting a therapeutic regimen. This might include using drugs with a higher risk profile where the likelihood of expected benefit has increased. Likewise, clinical investigators may desire to select for clinical trials a population with a high or low likelihood of obtaining a net benefit with a particular protocol. The methods described herein can be used by clinical investigators to select such a population. Thus, in some embodiments, the methods provide entry criteria and methods for selecting subjects for clinical trials, e.g., trials of AMD therapeutics, by selecting subjects having a given level of an AMD biomarker, e.g., CRP and/or IL-6 level, e.g., having a level that is above or below a reference, e.g., a reference indicated in Table A or B.

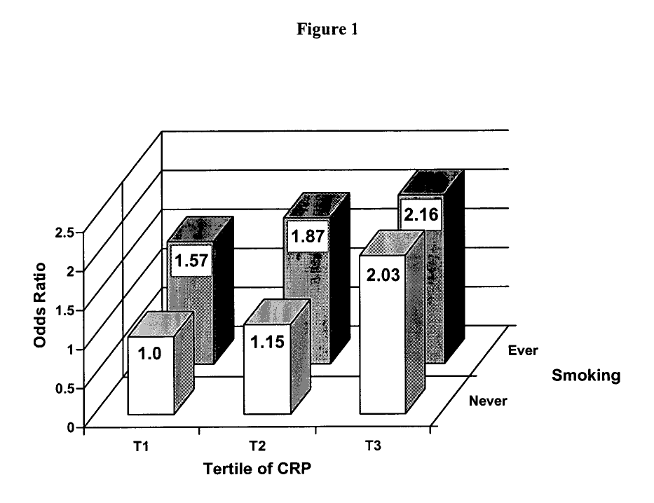

As described herein, it has been discovered that AMD biomarkers as described herein, including markers of systemic inflammation (e.g., CRP and/or IL-6), have predictive value independent of other known predictors of development or progression of AMD. Thus, the methods described herein do not involve simply duplicating a measurement that previously could have been made using other predictors. Instead, the markers of systemic inflammation provide information that is additive to previously known predictors. This is illustrated, for example, in Table 5, wherein the data were analyzed to characterize the risk profiles of subjects, taking into account both smoking history and levels of C-reactive protein. These data are illustrated in FIG. 1, which shows the relative risk of developing AMD associated with low, middle and high tertiles of total C reactive protein, and smoking. As is discussed in more detail below (see Example 1), the risk is additive.

Methods of Treatment

Also provided herein are methods for treating subjects with anti-inflammatory therapies to treat, prevent, or delay the development or progression of AMD. In some embodiments, an anti-inflammatory agent is administered to a subject who is known to have an above-normal level of a marker of systemic inflammation (e.g., CRP or IL-6); in some embodiments, the subject is otherwise free of overt symptoms calling for an anti-inflammatory agent, and/or is free of other symptoms of AMD.

In some embodiments, the anti-inflammatory agent is administered in conjunction with another modality for treating, preventing or delaying the development of AMD. A number of such treatments are known in the art, e.g., photodynamic therapy or laser photocoagulation to treat wet AMD, diet and exercise regiments, and/or vitamin supplements, e.g., as described in AREDS Research Group, Arch Ophthalmol 2001:119, 1417-1436; Seddon et al, JAMA, 1994; 272: 1413-1420, and U.S. Pat. No. 6,660,297.

In some embodiments, the methods include administering to the subject an anti-angiogenesis agent, e.g., anecortave acetate (Alcon), which reduces production of matrix metalloproteinase, a key agent in the growth of neovascular membranes; or an agent that inhibits vascular endothelial growth factor (VEGF). A number of inhibitors of VEGF signalling are known in the art and can include, e.g., ZD6474 (Tuccillo et al., Clin Cancer Res. 2005 Feb. 1; 11(3):1268-76); COX-2, Tie2 receptor, angiopoietin, and neuropilin inhibitors; pigment epithelium-derived factor (PEDF), endostatin, and angiostatin (21-25); VEGF inhibitory aptamers, e.g., Macugen™ (pegaptanib, Pfizer); antibodies or fragments thereof, e.g., anti-VEGF antibodies, e.g., bevacizumab (Avastin™, Genentech), or fragments thereof, e.g., ranibizumab (Lucentis™, Genetech); soluble fins-like tyrosine kinase 1 (sFlt1) polypeptides or polynucleotides (Harris et al., Clin Cancer Res. 2001 July; 7(7):1992-7; U.S. Pat. No. 5,861,484); PTK787/ZK222 584 (Maier et al., Graefes Arch Clin Exp Ophthalmol. 2005 Jan. 14; [Epub ahead of print]; KRN633 (Maier et al., Mol Cancer Ther. 2004 December; 3(12):1639-49); VEGF-Trap™ (Regeneron); intravitreal steroids, e.g., triamcinolone; and Alpha2-antiplasmin (Matsuno et al, Blood 2003; 120:3621-3628). For reviews of VEGF and its inhibitors, see, e.g., Campochiaro, Ocular neovascularisation and excessive vascular permeability, Expert Opin Biol Ther. 2004 September; 4(9):1395-402; Ferrara, Vascular Endothelial Growth Factor: Basic Science and Clinical Progress, Endocr. Rev., Aug. 1, 2004; 25(4):581-611; and Verheul and Pinedo, Vascular endothelial growth factor and its inhibitors, Drugs Today (Barc). 2003; 39 Suppl C:81-93.

Finally, triamcinolone acetate is another drug, currently in trials for the treatment of the neovascular stage of AMD, which can act as an anti-inflammatory agent and is administered by an intravitreal injection.

A number of diet and exercise modifications can be administered to reduce the risk of development of AMD or progression to advanced AMD. For example, decreased fat intake and increased intake of antioxidants, fish, and nuts have been shown to be related to decreased risk of developing AMD. See, e.g., Seddon et al., Arch Ophthalmol. 2003 June; 121(6):785-92, and Seddon et al., Arch Ophthalmol. 2003 December; 121(12):1728-37. Erratum in: Arch Ophthalmol. 2004 March; 122(3):426. A number of vitamin supplements are also available that are intended to enhance eye health, including vitamin A (e.g., as beta-carotene or lutein), vitamins C and E, and minerals zinc and copper, see, e.g., Age-Related Eye Disease Study Research Group, Arch Ophthalmol. 2001; 119:1417-1436.

In some embodiments, the methods include using a subject's levels of a biomarker as described herein, e.g., CRP and/or IL-6, to predict which subjects will be most likely to be responsive to treatment with an agent, e.g., an anti-inflammatory agent. In some embodiments, the methods include selecting (and, in some embodiments, administering) a particular treatment depending on the level of CRP and/or IL-6 or the other inflammatory markers in the subject.

Anti-inflammatory agents that can be used in the methods described herein include, but are not limited to, alclofenac; alclometasone dipropionate; algestone acetonide; alpha amylase; amcinafal; amcinafide; amfenac sodium; amiprilose hydrochloride; anakinra; anirolac; anitrazafen; apazone; balsalazide disodium; bendazac; benoxaprofen; benzydamine hydrochloride; bromelains; broperamole; budesonide; carprofen; cicloprofen; cintazone; cliprofen; clobetasol propionate; clobetasone butyrate; clopirac; cloticasone propionate; cormethasone acetate; cortodoxone; deflazacort; desonide; desoximetasone; dexamethasone dipropionate; diclofenac potassium; diclofenac sodium; diflorasone diacetate; diflumidone sodium; diflunisal; difluprednate; diftalone; dimethyl sulfoxide; drocinonide; endrysone; enlimomab; enolicam sodium; epirizole; etodolac; etofenamate; felbinac; fenamole; fenbufen; fenclofenac; fenclorac; fendosal; fenpipalone; fentiazac; flazalone; fluazacort; flufenamic acid; flumizole; flunisolide acetate; flunixin; flunixin meglumine; fluocortin butyl; fluorometholone acetate; fluquazone; flurbiprofen; fluretofen; fluticasone propionate; furaprofen; furobufen; halcinonide; halobetasol propionate; halopredone acetate; ibufenac; ibuprofen; ibuprofen aluminum; ibuprofen piconol; ilonidap; indomethacin; indomethacin sodium; indoprofen; indoxole; intrazole; isoflupredone acetate; isoxepac; isoxicam; ketoprofen; lofemizole hydrochloride; lomoxicam; loteprednol etabonate; meclofenamate sodium; meclofenamic acid; meclorisone dibutyrate; mefenamic acid; mesalamine; meseclazone; methylprednisolone suleptanate; morniflumate; nabumetone; naproxen; naproxen sodium; naproxol; nimazone; olsalazine sodium; orgotein; orpanoxin; oxaprozin; oxyphenbutazone; paranyline hydrochloride; pentosan polysulfate sodium; phenbutazone sodium glycerate; pirfenidone; piroxicam; piroxicam cinnamate; piroxicam olamine; pirprofen; prednazate; prifelone; prodolic acid; proquazone; proxazole; proxazole citrate; rimexolone; romazarit; salcolex; salnacedin; salsalate; salycilates; sanguinarium chloride; seclazone; sermetacin; sudoxicam; sulindac; suprofen; talmetacin; talniflumate; talosalate; tebufelone; tenidap; tenidap sodium; tenoxicam; tesicam; tesimide; tetrydamine; tiopinac; tixocortol pivalate; tolmetin; tolmetin sodium; triclonide; triflumidate; zidometacin; glucocorticoids; and zomepirac sodium.

Statins (HMG-CoA reductase inhibitors) are also considered to be anti-inflammatory agents (see, e.g., Curr Control Trials Cardiovasc Med 2000, 1:161-165) and can be used in the methods described herein. Statins include Pravachol™ (pravastatin, Bristol-Myers Squibb); Mevacor™ (Lovastatin, Merck); Zocor™ (simvastatin, Merck); Lescol™ (fluvastatin, Novartis); Lipitor™ (atorvastatin, Parke-Davis); Baycol™ (cerivastatin, Bayer); Crestor™ (rosuvastatin, Astra-Zeneca); and Advicor™ (lovastatin plus extended release niacin, Kos Pharmaceutical). In some embodiments, the anti-inflammatory agent is not a statin.

In some embodiments, the anti-inflammatory agent is aspirin.

The invention further provides compositions and kits including an anti-inflammatory agent and instructions (e.g., on a label or package insert such as instructions to the subject or to the clinician) for administering the anti-inflammatory agent to a subject to treat, prevent, and/or delay the development or progression of AMD. The anti-inflammatory agent can be in a pharmaceutical composition also including a pharmacologically acceptable carrier. As used herein, “pharmaceutically acceptable carrier” includes saline, solvents, dispersion media, coatings, antibacterial and antifungal agents, isotonic and absorption delaying agents, and the like, compatible with pharmaceutical administration. A pharmaceutical composition is typically formulated to be compatible with its intended route of administration, e.g., oral, mucosal, topical, transdermal, or parenteral. Supplementary active compounds can also be incorporated into the compositions. In some embodiments, the anti-inflammatory agent is in a form suitable for local delivery to the macular area, e.g., an implantable form.

An effective amount is a dosage of the anti-inflammatory agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health practitioner. For example, an effective amount can depend upon the degree to which a subject has abnormally elevated levels of markers of systemic inflammation. In some embodiments, the anti-inflammatory agents are used to prevent the development or progression of AMD, that is, they are used prophylactically in subjects at risk of developing AMD, or in subjects that already have AMD but whose AMD is likely to progress, e.g., to a more severe form of the disease. Thus, an “effective amount” is that amount which can lower the risk of, slow, or prevent altogether the development or progression of AMD. In some embodiments, the anti-inflammatory agent is administered in an effective amount, e.g., in an amount effective to reduce levels of one or more markers of systemic inflammation, e.g., to reduce the levels of the marker(s) to place the subject in a lower risk category, e.g., as described herein. The anti-inflammatory agent can be administered in one or more doses to achieve a desired therapeutic effect.

Generally, doses of active compounds can be from about 0.01 mg/kg per day to 1000 mg/kg per day. It is expected that doses ranging from 50-500 mg/kg will be suitable, typically administered orally, and in one to three (or more) administrations per day. Lower doses may result from other forms of administration, such as intravenous administration. In the event that a response in a subject is insufficient at the initial doses applied, higher doses (or effectively higher doses by a different, more localized delivery route) may be employed to the extent that subject tolerance permits. Multiple doses per day are contemplated to achieve appropriate systemic levels of compounds. The dosage and schedule will depend on the anti-inflammatory agent selected; a skilled practitioner would be able to select a regimen appropriate for the particular agent and subject. A number of anti-inflammatory agents are known in the art, and can be used in the methods described herein.

A variety of administration routes are available. The particular mode selected will depend upon the particular drug selected, the severity of the condition being treated and the dosage required for therapeutic efficacy. The methods described herein, generally speaking, can be practiced using any mode of administration that is medically acceptable, meaning any mode that produces effective levels of the active compounds without causing clinically unacceptable adverse effects. Such modes of administration include oral, rectal, topical, nasal, transdermal, or parenteral routes. The term “parenteral” includes subcutaneous, intravenous, intramuscular, or infusion. Local administration to the macular area can also be used. In some embodiments, the invention includes the use of implantable formulations, e.g., anti-inflammatory agents that are contained in a slow-release formula that can be implanted at or near the site of inflammation. Oral administration will typically be used for prophylactic and long term treatment because of the convenience to the subject as well as the dosing schedule. A number of oral compositions are known in the art and can be used in the methods described herein.

The delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for ex ample, U.S. Pat. No. 5,075,109.

Delivery systems can also include non-polymer systems that are: lipids including sterols such as cholesterol, cholesterol esters and fatty acids or neutral fats such as mono-, di- and tri-glycerides; hydrogel release systems; sylastic systems; peptide based systems; wax coatings; compressed tablets using conventional binders and excipients; partially fused implants; and the like. Specific examples include, but are not limited to, erosional systems in which the anti-inflammatory agent is contained in a form within a matrix such as those described in U.S. Pat. Nos. 4,452,775, 4,667,014, 4,748,034 and 5,239,660, and diffusional systems in which an active component permeates at a controlled rate from a polymer such as described in U.S. Pat. Nos. 3,832,253, and 3,854,480. Pump-based hardware delivery systems can be used, some of which are adapted for implantation. In addition, U.S. Pat. No. 6,331,313 describes a biocompatible ocular drug delivery implant device that can be used to deliver anti-inflammatory agents directly to the macular region.

Use of a long-term sustained release implant may be particularly suitable for treatment of chronic conditions. Long-term release means that the implant is constructed and arranged to delivery therapeutic levels of the active ingredient for at least 30 days, e.g., 60 days. Long-term sustained release implants are known to those in the art and include some of the release systems described herein.

Thus, the invention includes the use of an anti-inflammatory compound to treat, delay or prevent the development or progression of AMD. The invention further includes the use of an anti-inflammatory compound to modulate CRP and/or IL-6 levels, thereby treating, delaying or preventing the development or progression of AMD. Finally, the invention includes the use of an anti-inflammatory agent in the preparation of a medicament for use in the treatment of AMD.

The invention is further described in the following examples, which do not limit the scope of the invention described in the claims.

### EXAMPLES

### Example 1

### Association Between Markers of Systemic Inflammation and Age-Related Macular Degeneration (AMD)

The data described herein are a result of an ancillary study to the Age-Related Eye Disease Study (AREDS). AREDS is a prospective cohort study designed to assess the incidence, clinical course, prognosis and risk factors for AMD and cataract. Details of the AREDS design have been published elsewhere (see, e.g., AREDS Report No. 1. Control Clin Trials. 1999 December; 20(6):573-600; AREDS Report No. 3, Ophthalmology 2000; 107:2224 2232).

Study Population

For the present study, the Massachusetts Eye and Ear Infirmary (MEEI) and Devers Eye Institute (Devers) enrolled 1,026 subjects (517 and 509 respectively) into the AREDS clinical trial. Between January 1996 and April 1997, 930 subjects (91%) had a blood specimen drawn after randomization for this ancillary study, 465 at each clinic.

All but 1.3% of the specimens were obtained after fasting by the subject for at least 8 hours. Blood samples were processed immediately and then frozen in liquid nitrogen freezers at minus 140 degrees Centigrade. The study was approved by the Human Subjects' Committees of the two clinical centers and all subjects signed an informed consent statement.

Subjects were aged 55 to 80 at the time of enrollment. They were required to be in overall good health and were excluded if they had diseases with a poor seven-year survival prognosis (e.g., end stage cancer, advanced heart disease); hemochromatosis or Wilson's disease; oxalate kidney stone, alcoholism or drug abuse; or were unwilling or unable to discontinue non-study antioxidant vitamin or zinc supplementation. Subjects were also excluded if they had visual acuity of less than 20/32 in both eyes, advanced AMD, laser photocoagulation for AMD in both eyes, bilateral cataract extraction without signs of AMD, other eye diseases that would potentially compromise the evaluation of study outcomes, or if they used medications known to be toxic to the lens or retina.

Subjects were examined at six-month intervals, at which time information was collected on changes in visual acuity, disease incidence and progression, and risk factors from a visual acuity test, dilated lens and fundus examination, and a clinical interview. Baseline levels were established at the first visit. In addition, at the annual visit (occurring 12, 24, 36, etc. months after randomization), serum was drawn for specified AREDS tests, fundus and lens photographs were taken (except at the first annual visit), and a refraction was completed. Since the end of the clinical trial in April 2001, subjects have been examined annually.

Case-Control Definitions

Case-control definitions were adopted from a previous AREDS publication (8). According to reading center grading of fundus photographs at the visit most closely associated with the specimen draw, subjects in this ancillary study were divided into four maculopathy groups according to the size and extent of drusen in each eye, presence of geographic atrophy, and neovascular disease. These groups, numbered serially and based on increasing severity of drusen or type of AMD, were defined as follows.

Group 1 (No Drusen): (n=183) Each eye had no drusen or nonextensive small drusen, no pigment abnormalities, no advanced AMD, and no disqualifying ocular conditions. Most subjects had visual acuity of 20/32 or better in both eyes.

Group 2 (Intermediate Drusen): (n=200) At least one eye had one or more intermediate-size drusen, extensive small drusen, or pigment abnormalities associated with AMD. Neither eye had large drusen, advanced AMD, or a disqualifying ocular condition. Most subjects had visual acuity of 20/32 or better in both eyes.

Group 3 (Large Drusen or Intermediate AMD): (n=325) At least one eye had either one or more large sized drusen, about 20 intermediate-size soft drusen, or about 65 intermediate-size hard drusen. Neither eye had advanced AMD, a disqualifying ocular condition, or presence of geographic atrophy with diameter at least one eighth of that of the average disc, and most subjects had visual acuity of 20/32 or better in both eyes. Also included were subjects in whom one eye met these criteria and the fellow eye had either a disqualifying ocular condition or visual acuity of 20/32 or less not due to AMD.

Group 4 (Geographic Atrophy or Neovascular AMD—advanced AMD): (n=222) At least one eye had geographic atrophy definitely present (with diameter at least one eighth of that of the average disc; n=58) or neovascular AMD (further defined below; n=164). In most cases, there was a fellow eye with visual acuity of 20/32 or better with no evidence of advanced AMD or a disqualifying ocular condition.

Neovascular AMD included choroidal neovascularization or retinal pigment epithelial (RPE) detachment in one eye (non-drusenoid RPE detachment, serous sensory or hemorrhagic retinal detachment), subretinal hemorrhage, subretinal pigment epithelial hemorrhage, subretinal fibrosis, or evidence of confluent photocoagulation for neovascular AMD. The term “neovascular” is used as a summary term for this group of subjects, because most subjects in this group have direct evidence of choroidal neovascularization based on the assessment of fundus photographs. A few subjects in this group have serous RPE detachments.

The AREDS clinical trial (Age-Related Eye Disease Study Research Group, AREDS Report No. 8, Arch Ophthalmol. 2001; 119:1417-1436) showed that rates of progression to advanced AMD in Groups 1 and 2 were very low (5-year rates of 0.5% and 1.3%, respectively), and they were therefore combined here into one larger control group. For regression analyses, to enhance statistical power, Group 3 (5-year rate of about 18%) was combined with Group 4 (5-year rate of about 43%) to form the case group.

CRP Analysis

Serum samples were thawed and assayed for CRP. C-reactive protein was measured with a high-sensitivity assay as in studies of cardiovascular disease (Ridker et al., JAMA 2001; 285:2481-2485; Ridker, Circulation 2003; 107:363-369). The concentration of CRP was determined using an immunoturbidimetric assay on a Hitachi 911 analyzer (Roche Diagnostics, Indianapolis, Ind.), using reagents and calibrators from Denka Seiken (Niigata, Japan). In this assay, an antigen-antibody reaction occurs between CRP in the sample and an anti-CRP antibody that has been sensitized to latex particles, and agglutination results. The resulting antigen-antibody complex causes an increase in light scattering, which was detected spectrophotometrically, with the magnitude of the change being proportional to the concentration of CRP in the sample. This assay had a sensitivity of 0.003 mg/dL. The coefficients of variation of the assay at concentrations of 0.91, 3.07, and 1.338 mg/L were 2.81, 1.61, and 1.1%, respectively.

Apolipoprotein B Analysis

This assay was performed by an immunoturbidimetric technique on the Hitachi 911 analyzer (Roche Diagnostics—Indianapolis, Ind.), using reagents and calibrators from Wako (Wako Chemicals USA—Richmond, Va.). Polyclonal anti-apo B antibodies agglutinate with apo B present in the serum sample and form a complex. This agglutination was detected spectrophotometrically, with the magnitude of the change being proportional to the concentration of apoB in the sample. The day-to-day variabilities at apoB concentrations of 42.6, 88.3, and 132.8 mg/dL were 5.1, 3.9, and 4.0%, respectively.

Interleukin-6 (IL-6) Analysis

IL-6 was measured by an ultra-sensitive ELISA assay from R & D Systems. The assay employed the quantitative sandwich enzyme immunoassay technique. A monoclonal antibody specific for IL-6 was pre-coated onto a microtitre plate. After the addition of samples, standards, controls, and conjugates to the wells, IL-6 was sandwiched between the immobilized antibody and the enzyme-linked antibody specific to IL-6. Upon the addition of substrate, a color was generated that is proportional to the amount of IL-6 present in the sample. The minimum required volume for this assay was 200 μL. The assay had a sensitivity of 0.094 pg/mL, and the day-to-day variabilities of the assay at concentrations of 0.66, 1.97, and 8.16 pg/mL were 12.2, 7.6, and 9.9%, respectively.

Lipoprotein (a) Analysis

The concentration of Lp(a) was determined using a turbidimetric assay on the Hitachi 911 analyzer (Roche Diagnostics—Indianapolis, Ind.), using reagents and calibrators from Denka Seiken (Niigata, Japan). In this assay, an antigen-antibody reaction occurs between Lp(a) in the sample and an anti-Lp(a) antibody adsorbed to latex particles, and an agglutination results. This agglutination was detected spectrophotometrically, with the magnitude of the change being proportional to the concentration of Lp(a) in the sample. This method is the only commercial assay that is not affected by the Kringle Type 2 repeats (Marcovina S M et al. Use of a Reference Material Proposed by the International Federation of Clinical Chemistry and Laboratory Medicine to Evaluate Analytical Methods for the Determination of Plasma Lipoprotein(a). Clin Chem 2000; 46:1956-67). The day-to-day variabilities at Lp(a) concentrations of 17.6 and 58.1 mg/dL were 3.6 and 1.5%, respectively.

Soluble Intercellular Adhesion Molecule-1 (sICAM-1) Analysis

sICAM was measured by an ELISA assay (R & D Systems, Minneapolis, Minn.). The assay employed the quantitative sandwich enzyme immunoassay technique. A monoclonal antibody specific for sICAM-1 was pre-coated onto a microtitre plate. After the addition of samples, standards, controls and conjugates to the wells, sICAM was sandwiched between the immobilized antibody and the enzyme-linked antibody specific to sICAM. Upon the addition of substrate, a color was generated that is proportional to the amount of sICAM present in the sample. The minimum required volume for this assay was 25 uL. The assay had a sensitivity of 0.35 ng/mL and the day-to-day variabilities of the assay at concentrations of 64.2, 117, 290, and 453 ng/mL were 10.1, 7.4, 6.0 and 6.1%, respectively.

Soluble Vascular Cell Adhesion Molecule-1 (sVCAM-1) Analysis

sVCAM was measured by an ELISA assay (R & D Systems, Minneapolis, Minn.). The assay employed the quantitative sandwich enzyme immunoassay technique. A monoclonal antibody specific for sVCAM-1 was pre-coated onto a microtitre plate. After the addition of samples, standards, controls and conjugates to the wells, sVCAM was sandwiched between the immobilized antibody and the enzyme-linked antibody specific to sVCAM. Upon the addition of substrate, a color was generated that was proportional to the amount of sVCAM present in the sample. The minimum required volume for this assay was 25 μL. The assay had a sensitivity of 2.0 ng/mL. The day-to-day variabilities of the assay at concentrations of 9.8, 24.9, and 49.6 ng/mL were 10.2, 8.5, and 8.9%, respectively.

TNF-alpha Receptor II (TNF-R2) Analysis

TNF-R2 was measured by an ELISA assay from R & D Systems. The assay employed the quantitative sandwich enzyme immunoassay technique. A monoclonal antibody specific for TNF-R2 was pre-coated onto a microtitre plate. After the addition of samples, standards, controls and conjugates to the wells, TNF-R2 was sandwiched between the immobilized antibody and the enzyme-linked antibody specific to TNF-R2. Upon the addition of substrate, a color was generated that was proportional to the amount of TNF-R2 present in the sample. The minimum required volume for this assay was 50 uL. The day-to-day variabilities of the assay at concentrations of 89.9, 197, and 444 pg/mL were 5.1, 3.5, and 3.6%, respectively.

Risk Factor Definitions

Interviews to determine general risk factor and dietary information were conducted at the baseline (first) visit, and slitlamp biomicroscopy and ophthalmoscopy were performed at the time of blood drawing. The baseline risk factor variables that were considered in the analyses fall into five classes: demographic, medical, dietary/supplementation, use of medication, and ocular factors. For analysis, continuous variables (body-mass index, weight change from age 20 and sunlight exposure) were categorized by quartiles or tertiles based on the group without drusen (Group 1).

Demographic: The demographic variables included age, sex, race, education, and sunlight exposure (adult lifetime average annual ocular ultraviolet B exposure), adapted from McCarty et al (McCarty et al., Bull World Health Organ 1996; 74:353-360).

Medical: Medical variables included history of smoking, body mass index (BMI), weight change (increase or decrease) since age 20, hypertension (systolic>160 mmHg, diastolic>90 mmHg, or current use of antihypertensive medication), history of cardiovascular disease (at least one of the following: newly-developed heart disease after enrollment but prior to blood draw; occurrence of a stroke or myocardial infarction (developed after enrollment, but prior to blood draw); history of angina and taking an angina medication (e.g., dipyridamole, propranolol, beta-blocker, calcium-channel blocker, nitroglycerin, or isobide dinitrate); taking a heart disease medication (e.g., furosemide, ACE inhibitor, digoxin, blood thinning medication, cholesterol-lowering medication]), diabetes (e.g., under treatment for diabetes), and arthritis.

Dietary/Supplementation: The dietary/supplement variables included an antioxidant index and use of study treatment containing antioxidants. The antioxidant index was based on dietary results from a modified Block Food Frequency questionnaire (AREDS Manual of Operations) completed at the subject's baseline visit. Three measures were assessed: carotenoid intake (alpha-carotene, beta-carotene, lutein, lycopene, and beta-cryptoxanthin), vitamin C intake, and vitamin E intake. Subjects were grouped as having high antioxidant intake (above the highest quartile of intake for two out of the three measurements), low antioxidant intake (below the lowest quartile of intake for two out of the three measurements), or mixed antioxidant intake.

Subjects randomized to receive the study supplements containing high-dose antioxidants or high-dose antioxidants and zinc comprised the antioxidant treatment group. Subjects randomized to receive the study supplements containing zinc or placebo comprised those not in the antioxidant treatment group.

Use of Medication: Use of medication was defined as current use with five or more lifetime years of regular use. These medications included use of hydrochlorothiazide, diuretics (other than hydrochlorothiazide), aspirin, antacids, nonsteroidal anti-inflammatory drugs, thyroid hormones, beta-blockers, and estrogen and progesterone use (women).

Ocular: Ocular variables included iris color and refractive error. Iris color was graded at the reading center by comparing photographs of each eye with standards on a scale from 1 (light or blue) to 4 (dark or brown); a subject was ‘light’ if both eyes were code 1, ‘dark’ if both eyes were code 4, ‘mixed’ if at least one eye was code 2 or code 3 or eyes were not of the same code. A subject was ‘myopic’ if both eyes were myopic by −1.0 diopters spherical equivalent refractive error or more, ‘hyperopic’ if both eyes had +1.0 diopters spherical equivalent refractive error or more, or else ‘other’ which includes emmetropes and mixed cases.

Statistical Modeling and Analysis

The median values and interquartile ranges of CRP were calculated for each maculopathy group, and the most advanced AMD grade was compared with Group 1 using a non-parametric test of all p values. Conditional logistic regression analysis (SAS procedure LOGISTIC; version 8.02, SAS Institute, Inc. Cary, N.C.) was used to estimate odds ratios (ORs) and 95% confidence intervals (CIs) after the population was divided into quartile groups according to the quartile cut points of CRP values for the disease-free Group 1 (Ridker et al., JAMA 2001; 285:2481-2485; Ridker, Circulation 2003; 107:363-369). Prevalence odds ratios, which describe the association between disease and CRP (comparing cases in Groups 3 and 4 with controls in Groups 1 and 2), were computed for each CRP quartile group relative to the lowest quartile group. A test for linear trend was calculated based on the median levels of CRP within each quartile group.

Multivariate ORs were estimated from conditional logistic regression models, and were adjusted for age (57-65, 66-70, 71-83), sex, race, (white vs. other), smoking (ever smoked vs. never smoked), education (never completed high school, high school graduate, some college, or college graduate), body mass index (measured a weight in kilograms divided by height in meters squared; <23.9, 23.9-29.9, >29.9), antioxidant index (low, mixed, high), diabetes, history of cardiovascular disease (CVD), hypertension, and antioxidant treatment (taking study supplement containing antioxidants vs. taking study supplement containing no antioxidants). History of CVD and hypertension were not correlated. Other risk factors were evaluated as potential covariates but did not reach statistical significance in this ancillary study population (including weight change from age 20, sunlight exposure, arthritis, anti-inflammatory drugs, thyroid hormones, beta-blocker use, hormone use (women), iris color, and refractive error).

To evaluate a possible threshold effect, additional analyses were performed comparing cases with controls according to levels of CRP for Group 1 above and below the 90^{th} percentile, and above and below the mean CRP plus 2 standard deviations. Finally, to evaluate effect modification by cigarette smoking, additional logistic regression analyses were conducted to determine the ORs for AMD in 6 subgroups defined by never and ever smoking, and low, intermediate, and high tertiles of CRP.

Baseline Characteristics

Of the 930 subjects in this study, 61% were female and 39% were male, with a mean age of 69 years. Most of the subjects (71%) had some college or higher education. Forty-one percent of subjects had never smoked, 51% were former smokers, and 8% were current smokers. The median CRP value for all subjects was 2.7 mg/L, with a range of 0.2 to 117 mg/L, and the 90^{th} percentile range was 0.2 to 10.6 mg/l. CRP values did not differ according to age groups (55-65, 66-70, and 71+).

Table 1 displays the relationships between baseline characteristics and maculopathy groups, unadjusted for other variables. Significant differences (p<0.05) between subjects in maculopathy Groups 3 and 4 as compared to control subjects in Groups 1 and 2 included sex (lower proportion of females), smoking status (lower proportion of never smokers), and education (lower proportion with a college degree).

TABLE 1
Distribution of Baseline Characteristics by Maculopathy Group

| | Maculopathy Group | | | | | | | | | |
|---|---|---|---|---|---|---|---|---|---|---|
| | 1 | | 2 | | 3 | | 4 | | Total | |
| Characteristics | (n = 183) | | (n = 200) | | (n = 325) | | (n = 222) | | (N = 930) | |
| Mean (SD) age, y | 67 | (4.2) | 67 | (4.2) | 69 | (5.4) | 71 | (5.1) | 69 | (5.1) |
| Female | 116 | (64) | 143 | (72) | 184 | (57) | 122 | (55) | 565 | (61) |
| White race | 172 | (94) | 189 | (95) | 316 | (97) | 222 | (100) | 899 | (97) |

Smoking status

| | | | | | | | | | | |
|---|---|---|---|---|---|---|---|---|---|---|
| Never | 90 | (49) | 94 | (47) | 134 | (41) | 66 | (30) | 384 | (41) |
| Former | 77 | (42) | 93 | (47) | 170 | (52) | 131 | (59) | 471 | (51) |
| Current | 16 | (9) | 13 | (7) | 21 | (6) | 25 | (11) | 75 | (8) |

Body mass index

| | | | | | | | | | | |
|---|---|---|---|---|---|---|---|---|---|---|
| Bottom 25% | 46 | (25) | 37 | (19) | 77 | (24) | 50 | (23) | 210 | (23) |
| (≦23.9) | | | | | | | | | | |
| Middle 50% | 92 | (50) | 106 | (53) | 186 | (57) | 118 | (53) | 502 | (54) |
| (23.9-29.9) | | | | | | | | | | |
| Top 25% (≧29.9) | 45 | (25) | 57 | (29) | 62 | (19) | 54 | (24) | 218 | (23) |

Weight change from age of 20 years

| | | | | | | | | | | |
|---|---|---|---|---|---|---|---|---|---|---|
| Bottom 25% | 46 | (25) | 43 | (22) | 90 | (28) | 48 | (22) | 227 | (24) |
| (≦5.4 kg [12 lb]) | | | | | | | | | | |
| Middle 50% (5.9- | 92 | (50) | 96 | (48) | 158 | (49) | 106 | (48) | 452 | (49) |
| 19.8 [13-44 lb]) | | | | | | | | | | |
| Top 25% (≧19.8 | 45 | (25) | 61 | (31) | 77 | (24) | 68 | (31) | 251 | (27) |
| [44 lb]) | | | | | | | | | | |

Antioxidant index group

| | | | | | | | | | | |
|---|---|---|---|---|---|---|---|---|---|---|
| Low intake | 41 | (22) | 40 | (20) | 64 | (20) | 52 | (23) | 197 | (21) |
| Mixed intake | 106 | (58) | 114 | (57) | 190 | (58) | 131 | (59) | 541 | (58) |
| High intake | 36 | (20) | 46 | (23) | 71 | (22) | 39 | (18) | 192 | (21) |
| Assigned to | 92 | (50) | 91 | (46) | 166 | (51) | 113 | (51) | 462 | (50) |
| antioxidants | | | | | | | | | | |

Comorbidity

| | | | | | | | | | | |
|---|---|---|---|---|---|---|---|---|---|---|
| Hypertension | 63 | (34) | 67 | (34) | 112 | (34) | 87 | (39) | 329 | (35) |
| History of CVD^{§} | 47 | (26) | 42 | (21) | 73 | (22) | 71 | (32) | 233 | (25) |
| History of | 16 | (9) | 14 | (7) | 23 | (7) | 19 | (9) | 72 | (8) |
| diabetes | | | | | | | | | | |
| Arthritis | 75 | (41) | 96 | (48) | 157 | (48) | 107 | (48) | 435 | (47) |

Medication use

| | | | | | | | | | | |
|---|---|---|---|---|---|---|---|---|---|---|
| Anti- | 19 | (10) | 22 | (11) | 37 | (11) | 37 | (17) | 115 | (12) |
| inflammatory | | | | | | | | | | |
| drugs | | | | | | | | | | |
| Thyroid | 13 | (7) | 19 | (10) | 38 | (12) | 22 | (10) | 92 | (10) |
| hormones | | | | | | | | | | |
| β-Blocker use | 10 | (5) | 11 | (6) | 17 | (5) | 10 | (5) | 48 | (5) |

Iris color^{∥}

| | | | | | | | | | | |
|---|---|---|---|---|---|---|---|---|---|---|
| Light | 34 | (19) | 29 | (15) | 54 | (17) | 40 | (18) | 157 | (17) |
| Mixed | 128 | (70) | 149 | (75) | 243 | (75) | 160 | (72) | 680 | (73) |
| Dark | 21 | (11) | 21 | (11) | 28 | (9) | 22 | (10) | 92 | (10) |

Refractive error^{¶}

| | | | | | | | | | | |
|---|---|---|---|---|---|---|---|---|---|---|
| Myopic | 21 | (11) | 33 | (16) | 43 | (13) | 20 | (9) | 117 | (12) |
| Other | 87 | (48) | 75 | (38) | 152 | (47) | 111 | (50) | 425 | (46) |
| Hyperopic | 75 | (41) | 92 | (46) | 130 | (40) | 91 | (41) | 388 | (42) |

The data shown in Table 1 are No. (%) unless otherwise indicated. The antioxidant index is based on dietary results from a modified Block Food Frequency questionnaire (AREDS Manual of Operations). Three measures were assessed: carotenoid intake (alpha-carotene, beta-carotene, lutein, lycopene and beta-cryptoxanthin), vitamin C intake, and vitamin E intake. Subjects were grouped as having high antioxidant intake (above the highest quartile of intake for two out of the three measurements), low antioxidant intake (below the lowest quartile of intake for two out of the three measurements), or mixed antioxidant intake. Subjects assigned to take antioxidants took the study treatment antioxidant-containing formulation (vitamin C, vitamin E, beta-carotene).

History of cardiovascular disease was defined by at least one of the following: newly-developed heart disease after enrollment, but prior to blood draw; occurrence of a stroke or myocardial infarction after enrollment, but prior to blood draw; history of angina and taking an angina medication (dipyridamole, propranolol, beta-blocker, calcium-channel blocker, nitroglycerin, or isobide dinitrate); taking a heart disease medication (furosemide, ACE inhibitor, digoxin, blood thinning medication, cholesterol-lowering medication).

Iris color was graded on a scale of 1 (light or blue) to 4 (dark or brown); a subject was grouped as either light (if both eyes were code 1), dark (if both eyes were code 4) or mixed (at least one eye was code 2 or code 3 or eyes were not of the same code). A subject who was myopic by −1.0 diopters spherical equivalent refractive error or more was considered myopic; if both eyes had +1.0 diopters spherical equivalent refractive error, a subject was designated hyperopic; emmetropes and mixed cases are grouped as other.

### Example 2

### Association Between Maculopathy and C-Reactive Protein (CRP)

Median baseline plasma levels of the inflammatory marker CRP were higher among subjects who had more severe maculopathy (Table 2). The difference between the median value for the most advanced maculopathy Group 4 (3.4 mg/L) and the median for maculopathy group 1 (2.7 mg/L) was statistically significant (p=0.02).

TABLE 2
Levels of C-Reactive Protein (CRP) by Maculopathy Group

| | Maculopathy Group | | | | |
|---|---|---|---|---|---|
| | (P = 0.02 for nonparametric test of all CRP values | | | | |
| | (group 4 vs. 1)) | | | | |
| | 1 | 2 | 3 | 4 | Total |
| | (n = 183) | (n = 200) | (n = 325) | (n = 222) | (N = 930) |
| Median | 2.7 (1.2-6.5) | 2.4 (1.2-5.5) | 2.5 (1.2-5.7) | 3.4 (1.7-7.7) | 2.7 (1.4-6.1) |
| (interquartile | | | | | |
| range), mg/L | | | | | |
| Overall range, | 0.2-39.4 | 0.2-41.8 | 0.2-110.0 | 0.3-117.0 | 0.2-117.0 |
| mg/L | | | | | |

Table 3 displays the odds ratios for risk of AMD according to the quartile of CRP, for maculopathy case Groups 3 and 4 as compared with Groups 1 and 2, after adjustment for various other known and potential factors associated with AMD, using a non-parametric test of all CRP values. In an age- and sex-adjusted model, subjects above the highest quartile of CRP had higher risk of AMD (OR, 1.53; 95% confidence interval (CI), 1.03-2.28). The trend for an increase in risk of maculopathy with increase in CRP was statistically significant (p=0.02). After adjustment for additional covariates, the significant trend for an increase in risk remained for the highest quartile of CRP (OR, 1.65; 95% CI, 1.07-2.55, p for trend=0.02).

Odds ratios (ORs) were calculated by a comparison between maculopathy groups 3 and 4 (cases) and groups 1 and 2 (controls). P=0.02 for trend across quartiles (*). Statistics were adjusted for age (57-65, 66-70, and 71-83 years), sex, race (white vs. other), smoking (ever smoked vs. never smoked), education (never completed high school, high school graduate, some college, or college graduate), body mass index (<23.9, 23.9-29.9, >29.9), antioxidant index (low, mixed, high), diabetes, history of cardiovascular disease, hypertension, and antioxidant treatment (taking study supplement containing antioxidants vs. taking study supplement containing no antioxidants).

TABLE 3
Sample Sizes, CRP Levels, and ORs for Prevalence of Age-Related
Macular Degeneration According to Quartile of CRT

| CRP Quartile | | | |
|---|---|---|---|
| 1 | 2 | 3 | 4 |
| (0.2-1.2 mg/L) | (>1.2-2.7 mg/L) | (>2.7-6.5 mg/L) | (>6.5-117.0 mg/L) |

Sample size, No.

| | | | | |
|---|---|---|---|---|
| Cases | 142 | 118 | 142 | 145 |
| Controls | 106 | 102 | 90 | 85 |

Median CRP Level, mg/L (interquartile range)

| | | | | | | | | |
|---|---|---|---|---|---|---|---|---|
| Cases | 0.7 | (0.5-1.0) | 1.9 | (1.6-2.3) | 4.1 | (3.3-5.2) | 11.3 | (8.6-16.5) |
| Controls | 0.8 | (0.6-1.0) | 1.9 | (1.6-2.3) | 4.0 | (3.3-5.2) | 10.1 | (8.2-12.8) |
| Age- and sex- | 1.00 | | 1.06 | (0.72-1.54) | 1.23 | (0.84-1.82) | 1.53 | (1.03-2.28) |
| adjusted OR | | | | | | | | |
| (95% CI)* | | | | | | | | |

CI, confidence interval;
CRP, C-reactive protein;
OR, odds ratio.

Table 4 displays the association between CRP and maculopathy, using different cutpoints for values of CRP. Subjects with CRP levels above the 90th percentile had a significantly increased risk, with an OR of 1.75 (95% CI, 1.12-2.75) for the age- and sex-adjusted model and an OR of 1.92 (95% CI, 1.20-3.06) for the full multivariate model. Subjects with CRP values more than two standard deviations above the mean were also at increased risk with an OR of 1.89 (95% CI, 0.98-3.66) for the age- and sex-adjusted model and an OR of 2.03 (95% CI, 1.03-4.00) for the full multivariate model.

Odds ratios (ORs) were calculated by a comparison between maculopathy groups 3 and 4 (cases) and groups 1 and 2 (controls). P=0.02 for trend across quartiles (*). Statistics were adjusted for age (57-65, 66-70, and 71-83 years), sex, race (white vs. other), smoking (ever smoked vs. never smoked), education (never completed high school, high school graduate, some college, or college graduate), body mass index (<23.9, 23.9-29.9, >29.9), antioxidant index (low, mixed, high), diabetes, history of cardiovascular disease, hypertension, and antioxidant treatment (taking study supplement containing antioxidants vs. taking study supplement containing no antioxidants).

TABLE 4
Sample Sizes, CRP Levels, and ORs for Prevalence of Age-Related
Macular Degeneration According to Cut Points of CRP

| 90th Percentile of CRP | | Mean CRP + 2 SDs | |
|---|---|---|---|
| Below | Above | Below | Above |
| (0.2-10.6 mg/L) | (>10.6-117.0 mg/L) | (0.2-16.8 mg/L) | (>16.8-117.0 mg/L) |

Sample size, No.

| | | | | |
|---|---|---|---|---|
| Cases | 474 | 73 | 513 | 34 |
| Controls | 350 | 33 | 369 | 14 |

Median CRP level, mg/L (interquartile range)

| | | | | | | | | |
|---|---|---|---|---|---|---|---|---|
| Cases | 2.4 | (1.2-4.8) | 16.3 | (14.0-26.2) | 2.7 | (1.3-5.6) | 27.6 | (19.6-41.6) |
| Controls | 2.2 | (1.1-4.4) | 14.5 | (11.2-19.9) | 2.4 | (1.2-5.1) | 23.6 | (18.8-34.8) |
| Age- and sex- | 1.00 | | 1.75 | (1.12-2.75) | 1.00 | | 1.89 | (0.98-3.66) |
| adjusted OR | | | | | | | | |
| (95% CI) | | | | | | | | |
| Multivariate- | 1.00 | | 1.92 | (1.20-3.06) | 1.00 | | 2.03 | (1.03-4.00) |
| adjusted OR | | | | | | | | |
| (95% CI) | | | | | | | | |

CI, confidence interval;
CRP, C-reactive protein;
OR, odds ratio.

These results demonstrate that elevated CRP levels are associated with risk of developing AMD. Thus, CRP levels can be used to predict a subject's risk of developing AMD.

### Example 3

### Effect Modification by Smoking

To determine whether the effect of CRP was modified by cigarette smoking, a consistently strong risk factor for AMD, OR's were computed for AMD in analyses in which subjects were stratified into six groups according to smoking (ever or never) and tertile of CRP, as shown in Table 5.

TABLE 5
Sample Sizes, CRP Levels, and ORs for Prevalence of Age-Related Macular
Degeneration According to Tertile of CRP and Smoking Status

| Tertile Groups of CRP | | | | | |
|---|---|---|---|---|---|
| 1 (0.2-1.7 mg/L) | | 2 (>1.7-4.5 mg/L) | | 3 (>4.5-117.0 mg/L) | |
| Never | Ever | Never | Ever | Never | Ever |
| Smoker | Smoker | Smoker | Smoker | Smoker | Smoker |

Sample size, No.

| | | | | | | |
|---|---|---|---|---|---|---|
| Cases | 67 | 105 | 61 | 109 | 72 | 133 |
| Controls | 72 | 69 | 65 | 59 | 47 | 71 |

Median CRP levels, mg/L (interquartile range)

| | | | | | | |
|---|---|---|---|---|---|---|
| Cases | 0.9 (0.6-1.3) | 0.9 (0.6-1.4) | 2.5 (2.1-3.2) | 2.9 (2.3-3.4) | 9.7 (5.5-14.2) | 8.6 (6.0-14.4) |
| Controls | 1.1 (0.8-1.5) | 0.9 (0.6-1.4) | 2.6 (2.1-3.1) | 2.9 (2.2-3.5) | 7.7 (5.7-11.0) | 9.0 (6.4-10.6) |
| Multivariate- | 1.00 | 1.57 (0.97-2.55) | 1.15 (0.69-1.93) | 1.87 (1.15-3.06) | 2.03 (1.19-3.46) | 2.16 (1.33-3.49) |
| adjusted OR | | | | | | |
| (95% CI) | | | | | | |

CI, confidence interval;
CRP, C-reactive protein;
OR, odds ratio.

Odds ratios were adjusted for age (57-65, 66-70, and 71-83 years), sex, race (white vs. other), smoking (ever smoked vs. never smoked), education (never completed high school, high school graduate, some college, or college graduate), body mass index (<23.9, 23.9-29.9, >29.9), antioxidant index (low, mixed, high), diabetes, history of cardiovascular disease, hypertension, and antioxidant treatment (taking study supplement containing antioxidants vs. taking study supplement containing no antioxidants).

For smokers and never-smokers, higher levels of CRP were associated with higher risk of AMD. Subjects in the high risk group (current and past smokers with the highest level of CRP) had a statistically significant 2.16-fold higher risk (95% CI, 1.33-3.49) of maculopathy compared with the low risk group (those who never smoked and had the lowest CRP level), after adjustment for other factors. In light of the clinical observation that many subjects who do not smoke nonetheless develop maculopathy, it is interesting to note that among never smokers, the odds of developing AMD were 2.03 in the highest tertile of CRP (95% CI 1.19, 3.46), compared to those in the lowest quartile of CRP, after adjustment for other factors. To evaluate this relationship further, the effect of smoking (ever or never) stratified by tertile of CRP was analyzed. Cigarette smoking increased risk of AMD more than 1.7 fold in the lower two tertiles of CRP ORs 11.79 (95% CI, 1.06-3.00) and 1.90 (95% CI, 1.12-3.22), but there was no association between smoking and CRP in the highest level of CRP (OR 1.02).

These results demonstrate that the highest levels of CRP appear to increase risk of developing AMD, independent of smoking. Thus, CRP levels can be used to predict a subject's risk of developing AMD.

### Example 4

### Association Between Inflammatory and Lipid Biomarkers and Progression to Advanced AMD

In the prospective study described in this Example, the relative risk of development of more advanced AMD was evaluated.

Study Population

The study population used in this Example consisted of patients with nonexudative AMD and best corrected visual acuity of 20/200 or better in at least one eye, and aged 60 years and older at baseline. Other inclusion criteria included willingness to participate in a long term study that involved annual dilated eye examinations and fundus photography. Patients were excluded if they were unable to speak English or had decreased hearing or cognitive function such that they may not fully understand a health status and dietary interview. All patients were examined at the Massachusetts Eye and Ear Infirmary, Boston.

Of the 397 persons who were eligible for enrollment between July 1989 and May 1998, 366 (92%) were enrolled. The Human Subjects Committee at the Massachusetts Eye and Ear Infirmary approved the study, and all subjects signed a consent form to participate. Of the 366 participants enrolled, 36 were not considered for analyses because of inability to complete the initial study examination (n=5), lack of follow up data (n=17), or lack of one or more primary independent variables (n=14). To conduct the analyses, men and women were excluded who reported, at baseline, ever having a diagnosis of cancer (n=61) (except nonmelanoma skin cancer), which could influence the dietary and other variables assessed in these analyses. Finally, to maintain the quality of data for these analyses, 8 individuals were excluded whose dietary questionnaires had inadequate or missing answers (n=6) or extreme values (n=2). Participants whose responses were not included in the analyses (n=8) did not differ significantly by age, sex, or education from those who were included. Of the 261 individuals, 251 had biomarkers assessed, and were included in these analyses. The methodology for this study was described previously; see Seddon et al., Arch Ophthalmol. 2003 June; 121(6):785-92 and Seddon et al., Arch Ophthalmol. 2003 December; 121(12):1728-37.

Data Collection and Classification of AMD

Information was collected from various sources including a fasting blood specimen, a standardized risk factor questionnaire that was administered over the telephone by a trained interviewer, a clinical interview, a validated food frequency questionnaire (Ajani et al., Invest. Ophthal. Vis. Sci. 1994; 35:2725-2733), and measurements of height, weight, and blood pressure. Body mass index during the initial examination was calculated as weight in kilograms divided by the height in meters squared. At baseline and at each annual visit, a complete dilated eye examination was performed, including a refraction, assessment of best corrected visual acuity, a lens evaluation, and slit-lamp examination of the macula. Stereoscopic color fundus photographs of the macula were also obtained.

Macular characteristics were graded within a 3000 pm radius centered on the foveal center. The 5 grade classification scale of AMD was used, as described in the “Clinical Age-Related Maculopathy Staging System,” (Seddon et al., IOVS, 1997; 3172, S676, and Afshari, Sharma, Seddon, presented at American Academy of Ophthalmology meeting, 2000; Seddon et al., Arch Ophthalmol. 2003; 121: 1728-1737; Seddon et al., Arch Ophthalmol. 2003; 121:785 792; and Seddon et al., Am J Hum. Genet 2003; 73: 780-790), which was modified from the Age-Related Eye Disease Study (AREDS Report No. 8, Arch Ophthalmol. 2001; 119:1417 1436) and evaluated for reliability (Seddon et al., Arch Ophthalmol. 2003; 121:1728-1737; and Seddon et al., Arch Ophthalmol. 2003; 121:785 792). Eyes with extensive small drusen (≧15 small drusen; <63 um), non-extensive intermediate drusen (<20 drusen; ≧63 um but <125 um), or pigment abnormalities associated with AMD were assigned a grade of 2. Eyes with extensive intermediate or large (≧125 um) drusen were assigned a grade of 3. Eyes with geographic atrophy received a grade of 4. If there was evidence of retinal pigment epithelial detachment or choroidal neovascular membrane, a grade of 5 was assigned. Eyes received a grade of 1 if none of these signs was present. Advanced AMD was defined as grades 4 and 5.

Biomarker Analyses

Fasting blood specimens were all drawn in the morning, at the baseline visit, and were processed immediately and then frozen in liquid nitrogen freezers until analysis. All assays but the cytokines and adhesion molecules were measured using a Hitachi 911 chemistry analyzer (Roche Diagnostics, Indianapolis, Ind.). CRP was measured using a high sensitivity assay from Denka Seiken (Niigata, Japan). IL-6, TNF-R2, ICAM-1, and VCAM-1 were measured by ELISA methods from R & D Systems, Minneapolis, Minn. Lp(a) concentration was measured using an assay from Denka Seiken that is not affected by the Kringle Type 2 repeats (Marcovina et al., Clin. Chem. 2000; 46:1956-67). TNF-R2 was used in our analysis to measure the presence of inflammation, because it has been shown to be more stable and requires less blood than TNF-alpha; thus making it more reliable as an indicator of disease. Apo B assay was performed by an immunoturbidimetric technique using reagents from Wako (Wako Chemicals USA, Richmond, Va.).

Statistical Analyses

Progression to advanced AMD was defined either as one eye progressing from a grade of less than 4 to grades 4 or 5 or progressing from grade 4 to grade 5 at any follow up visit. Although both grades 4 and 5 are classified as advanced disease, eyes with geographic atrophy (grade 4) can progress to neovascular disease (grade 5). Sunness et al. (Ophthalmology 1999; 106:910-919) reported 4 year conversion rates of 11% for individuals with bilateral atrophy, 34% for those with unilateral atrophy and choroidal neovascular membrane in the fellow eye, and 19% rate of progression overall. They also noted that the development of choroidal neovascular membrane had a negative effect on the degree of visual acuity loss. Therefore, progression from atrophy to choroidal neovascular membrane (grade 4 to 5) was also included as an outcome.

Regression was not considered in the analyses. Each subject was considered to have progressed only once during the follow up period, counting the first eye that progressed. The rationale for this definition of progression was that AMD is a progressive disease and regression at this advanced stage is uncommon. Specifically, among 107 people who progressed from baseline, only 2 individuals regressed at a subsequent visit; both of these subjects then subsequently progressed to advanced AMD.

First, age-adjusted analyses were performed relating progression of disease to CRP levels, adjusting for age-sex groups coded as dummy variables (men aged 60 to 69 years, men aged 70 to 79 years, men aged 80 years and older, women aged 60 to 69 years, women aged 70 to 79 years, and women aged 80 years and older) and energy intake by including log total energy intake (calories) as an additional covariate. The principal method of analysis was the Cox proportional hazards model. An adjusted relative risk (RR) of progression was computed for sex-specific quartiles 2 to 4 of CRP vs sex-specific quartile 1 after controlling for age-sex group, energy (log), and protein intake (quartiles). The full multivariate model also included the number of years of education (<12 or ≧12), smoking status (current, past, or never), body mass index (<25, 25 to 29, or ≧30 kg/m²), systolic blood pressure (analyzed continuously), cardiovascular disease, log energy (continuous), protein intake (quartile), energy-adjusted log beta-carotene intake (continuous), self-reported alcohol intake (grams per day as a continuous variable), physical activity (number of times per week of vigorous physical activity as a continuous variable), and initial AMD grade (1-5, categorical). Tests for trend were also conducted corresponding to the adjusted and multivariate RRs by substituting a single trend variable of CRP coded as 1 to 4 and interpreted as a continuous variable. Ninety-five percent confidence intervals (CIs) were computed for the multivariate RRs, and 2-sided p values were computed for all models. A similar approach was used to model the relationship between progression of AMD and the other biomarkers: 1′-6, TNF-R2, ICAM-1, VCAM-1, Lp(a), and ApoB. Furthermore, the analyses concerning biomarkers were repeated, adjusting for the individual nutrients reported to be beneficial in the Age-Related Eye Disease Study: total intake of zinc and vitamins C and E from food and supplements (AREDS Report No. 8. Arch Ophthalmol. 2001; 119:1417 1436).

Finally, to better understand the effect of other AMD risk factors on CRP and IL-6, linear regression analyses were performed, for log CRP and log IL-6 on all other covariates in Tables 6-8. Furthermore, rates of progression to advanced AMD for various levels of CRP and IL-6 were calculated. All analyses were performed using SAS software, version 8.0 (SAS Institute Inc, Cary, N.C.).

Results

Two hundred fifty one participants (96 males, 155 females) were included in the final analyses with a mean age of 72 years. Almost all subjects (99.6%) were Caucasian. The average follow-up time was 4.6 years. Of the 251 participants with AMD, 96 patients progressed to advanced stages of disease.

Tables 6-8 display the relationships between baseline characteristics and the various biomarkers, unadjusted for other variables. Physical activity was inversely related to CRP, IL-6 and TNF-R2 but positively related to VCAM-1. Current smoking had a positive association with all 7 biomarkers. Systolic blood pressure was positively related CRP, IL-6, TNF-R2, ICAM-1, VCAM-1, and ApoB while Lp(a) showed only a slight positive relationship with systolic blood pressure. CVD was positively related to CRP, IL-6, TNF-R2, ICAM-1, VCAM-1 and Lp(a). Fish intake was inversely related to CRP and IL-6. BMI was positively related to CRP, TNF-R2, and VCAM-1. Alcohol was inversely related to TNF-R2 and VCAM. Energy intake showed a positive association with Lp(a). Beta-carotene was inversely related to CRP, IL-6, VCAM-1, and Lp(a). Zinc was inversely related to IL-6 and ApoB. Vitamin C was inversely related to CRP and IL-6. Vitamin E was inversely related to IL-6 and ApoB.

In Tables 6-8, intake of beta carotene, zinc, vitamin C, and vitamin E are expressed as a geometric mean after sex-specific calorie-adjustment; other values are means or percents. Education is percent with at least high school education. Physical activity is the mean number of times/week of vigorous activity.

TABLE 6
Characteristics of Progression Study Population by Quartiles of CRP, IL-6, and TNF-αR

| | QUARTILES | | | | | | | | | | | |
|---|---|---|---|---|---|---|---|---|---|---|---|---|
| | | | | | | | | | TNF-Alpha Receptor | | | |
| | CRP | | | | IL-6 | | | | II | | | |
| Biomarker | 1 | 2 | 3 | 4 | 1 | 2 | 3 | 4 | 1 | 2 | 3 | 4 |
| N | 62 | 61 | 65 | 63 | 61 | 61 | 64 | 63 | 60 | 62 | 64 | 63 |
| Age (y) | 72 | 73 | 72 | 73 | 71 | 72 | 72 | 74 | 70 | 72 | 72 | 75 |
| Male (%) | 39 | 38 | 38.5 | 38 | 38 | 38 | 37.5 | 38 | 37 | 39 | 37.5 | 38 |
| Education (%) | 87 | 87 | 85 | 92 | 92 | 80 | 91 | 87 | 92 | 84 | 87.5 | 87 |
| Initial Worse Eye | | | | | | | | | | | | |
| Grade 2 | 10 | 8 | 9 | 8 | 10 | 7 | 8 | 9 | 13 | 4 | 6 | 11 |
| Grade 3 | 27 | 23 | 22 | 22 | 31 | 21 | 21 | 22 | 25 | 30 | 21 | 19 |
| Grade 4 | 4 | 11 | 10 | 13 | 3 | 15 | 9 | 11 | 6 | 12 | 10 | 10 |
| Grade 5 | 21 | 19 | 24 | 20 | 17 | 18 | 26 | 21 | 16 | 16 | 27 | 23 |
| Physical Activity | 2.2 | 1.5 | 1.9 | 1.2 | 2.1 | 2.0 | 1.7 | 1.0 | 2.1 | 1.8 | 1.7 | 1.2 |
| (mean) | | | | | | | | | | | | |
| Current smoker | 2 | 8 | 11 | 16 | 7 | 10 | 8 | 13 | 8 | 8 | 9 | 11 |
| (%) | | | | | | | | | | | | |
| Past Smoker (%) | 50 | 64 | 58.5 | 52 | 54 | 56 | 55 | 62 | 65 | 52 | 52 | 59 |
| Systolic BP | 134 | 138 | 142 | 140 | 133 | 140 | 143 | 139 | 131 | 138 | 142 | 144 |
| (mmHg) | | | | | | | | | | | | |
| (mean) | | | | | | | | | | | | |
| Cardiovascular | 16 | 23 | 18 | 22 | 13 | 21 | 19 | 25 | 15 | 21 | 19 | 24 |
| Disease | | | | | | | | | | | | |
| (Yes) (%) | | | | | | | | | | | | |
| Fish Intake (%) | | | | | | | | | | | | |
| <1/wk | 35.5 | 36 | 32 | 41 | 38 | 38 | 28 | 41 | 42 | 34 | 31 | 38 |
| ≧1/wk, <2/wk | 29 | 25 | 40 | 35 | 23 | 34 | 36 | 35 | 23 | 34 | 39 | 32 |
| ≧2/wk | 35.5 | 39 | 28 | 24 | 39 | 28 | 36 | 24 | 35 | 32 | 30 | 30 |
| BMI, Kg/m² | 26 | 27.5 | 28.5 | 28 | 26 | 28 | 28 | 28 | 26 | 27 | 28.5 | 29 |
| Alcohol, g/day | 7 | 7 | 8.5 | 7 | 9 | 8 | 6 | 8 | 12 | 6 | 7 | 5 |
| Calories | 1466 | 1337 | 1443 | 1317 | 1398 | 1349 | 1446 | 1342 | 1306 | 1427 | 1346 | 1458 |
| Beta carotene | 3875 | 3832 | 2962 | 3243 | 3787 | 3455 | 3603 | 3141 | 3320 | 3970 | 2832 | 3971 |
| intake* (μg/day) | | | | | | | | | | | | |
| Zinc* (mg/day) | 15 | 17 | 16 | 15 | 19 | 15 | 16 | 14 | 17 | 14 | 17 | 15 |
| Vitamin C* | 271 | 235 | 218 | 174 | 257 | 215 | 230 | 192 | 237 | 222 | 201 | 231 |
| (mg/day) | | | | | | | | | | | | |
| Vitamin E* | 23 | 37 | 22 | 18 | 32 | 23 | 31 | 15 | 31 | 26 | 18 | 24 |
| (mg/day) | | | | | | | | | | | | |

TABLE 7
Characteristics of Study Population by Quartiles of ICAM, VCAM, and LP(a)

| | QUARTILE | | | | | | | | | | | |
|---|---|---|---|---|---|---|---|---|---|---|---|---|
| | ICAM | | | | VCAM | | | | LP(a) | | | |
| Biomarker | 1 | 2 | 3 | 4 | 1 | 2 | 3 | 4 | 1 | 2 | 3 | 4 |
| N | 61 | 62 | 63 | 63 | 60 | 63 | 63 | 63 | 61 | 64 | 64 | 62 |
| Age (y) | 72 | 72 | 71 | 73 | 71 | 72 | 72 | 74 | 73 | 71.5 | 73 | 72 |
| Male (%) | 36 | 39 | 38 | 38 | 63 | 62 | 62 | 62 | 38 | 39 | 37.5 | 39 |
| Education (%) | 95 | 84 | 83 | 89 | 93 | 87 | 87 | 82.5 | 88.5 | 97 | 87.5 | 77 |
| Initial Worse | | | | | | | | | | | | |
| Eye | | | | | | | | | | | | |
| Grade 2 | 8 | 10 | 7 | 9 | 11 | 3 | 10 | 10 | 6 | 7 | 15 | 7 |
| Grade 3 | 23 | 23 | 31 | 18 | 26 | 29 | 26 | 14 | 20 | 28 | 21 | 25 |
| Grade 4 | 10 | 7 | 10 | 11 | 7 | 11 | 6 | 14 | 15 | 12 | 1 | 10 |
| Grade 5 | 20 | 22 | 15 | 25 | 16 | 20 | 21 | 25 | 20 | 17 | 27 | 20 |
| Physical | 1.6 | 1.9 | 1.4 | 1.9 | 1.8 | 1.8 | 1.9 | 1.3 | 1.6 | 1.6 | 2.2 | 1.2 |
| Activity (mean) | | | | | | | | | | | | |
| Current smoker | 2 | 10 | 16 | 10 | 8 | 10 | 14 | 5 | 10 | 12.5 | 3 | 11 |
| (%) | | | | | | | | | | | | |
| Past Smoker (%) | 59 | 52 | 51 | 65 | 57 | 51 | 54 | 65 | 57 | 52 | 64 | 52 |
| Systolic BP | 138 | 136 | 139 | 142 | 133 | 139 | 139 | 144 | 142 | 137 | 137 | 138 |
| (mmHg) (mean) | | | | | | | | | | | | |
| Cardiovascular | 11.5 | 21 | 17.5 | 29 | 10 | 24 | 19 | 25 | 13 | 19 | 25 | 23 |
| Disease | | | | | | | | | | | | |
| (Yes) (%) | | | | | | | | | | | | |
| Fish Intake (%) | | | | | | | | | | | | |
| <1/wk | 31 | 39 | 36.5 | 38 | 40 | 35 | 36.5 | 33 | 34 | 41 | 23 | 47 |
| ≧1/wk, <2/wk | 31 | 29 | 38 | 30 | 25 | 33 | 33 | 36.5 | 38 | 27 | 34 | 31 |
| ≧2/wk | 38 | 32 | 25 | 32 | 35 | 32 | 30 | 30 | 28 | 33 | 42 | 23 |
| BMI, Kg/m² | 27 | 28 | 28 | 28 | 26 | 27 | 28 | 29 | 28 | 27 | 27 | 28 |
| Alcohol, g/day | 8 | 7.5 | 7 | 7 | 10.6 | 7 | 7 | 5 | 7.28 | 11 | 5.8 | 4.9 |
| Calories | 1374 | 1360 | 1375 | 1426 | 1413 | 1347 | 1323 | 1456 | 1338 | 1348 | 1411 | 1466 |
| Beta carotene | 3115 | 4229 | 3444 | 3255 | 3928 | 3531 | 3225 | 3323 | 3701 | 3649 | 3310 | 3160 |
| intake* (μg/day) | | | | | | | | | | | | |
| Zinc* (mg/day) | 17 | 14 | 15 | 17 | 15 | 15 | 15 | 17 | 13 | 17 | 16 | 16 |
| Vitamin C* | 210 | 242 | 219 | 219 | 238 | 195 | 238 | 221 | 208 | 215 | 241 | 223 |
| (mg/day) | | | | | | | | | | | | |
| Vitamin E* | 30 | 31 | 16 | 23 | 28 | 21 | 21 | 28 | 21 | 29 | 23 | 23 |
| (mg/day) | | | | | | | | | | | | |

TABLE 8
Characteristics of Study Population by Quartiles of ApoB

| | QUARTILES | | | |
|---|---|---|---|---|
| | ApoB | | | |
| Biomarker | 1 | 2 | 3 | 4 |
| N | 62 | 64 | 62 | 63 |
| Age (y) | 73 | 71 | 72 | 72 |
| Male (%) | 37 | 37.5 | 40 | 38 |
| Education (%) | 92 | 86 | 87 | 86 |
| Initial Worse Eye | | | | |
| Grade 2 | 8 | 7 | 10 | 10 |
| Grade 3 | 16 | 29 | 30 | 19 |
| Grade 4 | 13 | 8 | 10 | 7 |
| Grade 5 | 25 | 20 | 12 | 27 |
| Physical Activity (mean) | 1.4 | 2.2 | 1.8 | 1.3 |
| Current smoker (%) | 8 | 3 | 13 | 13 |
| Past Smoker (%) | 60 | 48 | 56 | 60 |
| Systolic BP (mmHg) (mean) | 137 | 138 | 140 | 140 |
| Cardiovascular Disease (Yes)(%) | 16 | 22 | 21 | 21 |
| Fish Intake | | | | |
| <1/wk | 47 | 31 | 29 | 38 |
| ≧1/wk, <2/wk | 24 | 36 | 35.5 | 33 |
| ≧2/wk | 29 | 33 | 35.5 | 29 |
| BMI, Kg/m² | 27 | 27 | 29 | 28 |
| Alcohol, g/day | 7 | 8 | 4 | 10 |
| Calories | 1375 | 1443 | 1451 | 1296 |
| Beta carotene intake (μg/day) | 3688 | 3584 | 2937 | 3629 |
| Zinc (mg/day) | 18 | 15 | 15 | 14 |
| Vitamin C (mg/day) | 249 | 233 | 193 | 216 |
| Vitamin E (mg/day) | 33 | 20 | 22 | 23 |

Association Between Biomarkers and Progression of AMD

Relative risks (RRs) for progression to advanced AMD by quartiles of inflammatory biomarkers are shown in Table 9. The “Adjusted RR” is adjusted for age-sex group (60-69 Male/70-79 Male/80+Male/60-69 Female/70-79 Female/80+Female), log calories (continuous), and protein intake (quartile). The Multivariate RR (model 1) is adjusted for age-sex group (60-69 Male/70-79 Male/80+Male/60-69 Female/70-79 Female/80+Female), education (≧high school vs. <high school), smoking (current/past/never), BMI (<25/25-29.9/30+), systolic blood pressure, cardiovascular disease, log calories (continuous), protein intake (quartile), calorie-adjusted beta carotene intake (continuous), alcohol intake (continuous), physical activity (continuous-times/wk vigorous), and initial AMD grade (categorical). The Multivariate RR (model 2) is adjusted for the variables in model 1 plus total intake of zinc, vitamin C and vitamin E.

The highest quartile of CRP was positively related to progression of AMD, with a two-fold greater risk compared with the lowest level of CRP after controlling for covariates (RR 2.10, 95% CI 1.06-4.18). After further adjustment for antioxidant nutrients, the effect was essentially unchanged, with a RR of 2.02 (95% CI 1.06-4.18). The P values for trend for an increasing risk of progression with higher levels of CRP were 0.046 and 0.057, respectively. Similarly, the highest quartile of IL-6 was related to progression of AMD (multivariate RRs of 1.81, 95% CI 0.97-3.36, and 1.96, 95% CI 1.04-3.70), compared with the lowest quartile. The trend for a higher risk of progression of AMD with higher levels of IL-6 was statistically significant for both models (P values for trend=0.026, 0.017, respectively).

For both CRP and IL-6, the attenuation of the effect compared to the simple adjusted model indicates the presence of positive confounding factors, most likely smoking and body mass index. Both of these variables are positively related to AMD (Seddon et al., JAMA. 1996; 276:1141-1146; Smith et al., Ophthalmology. 2001; 108:697 704; Seddon et al., Arch Ophthalmol. 2003; 121:785 792; and AREDS Report No. 3. Ophthalmology 2000; 107:2224-2232) and positively correlated with CRP and IL-6, as described below. For TNF-R2, although weakly associated with progression in age and gender-adjusted analyses, the effect became non-significant after multivariate adjustment for known risk factors for AMD. No association was seen with ICAM-1 (date not shown), but VCAM-1 showed a slight, non-significant trend for a positive association with progression of AMD comparing the highest with the lowest quartiles (multivariate RR 1.94, 95% CI 0.99-3.80, and RR.1.76, 95% CI 0.89-3.47, respectively). The relative risks for progression to advanced AMD according to quartiles of the lipid biomarkers was also evaluated. There was no association with Lp(a) and a weak positive association with ApoB (RR 1.40 for both multivariate models), which was not significant (data not shown).

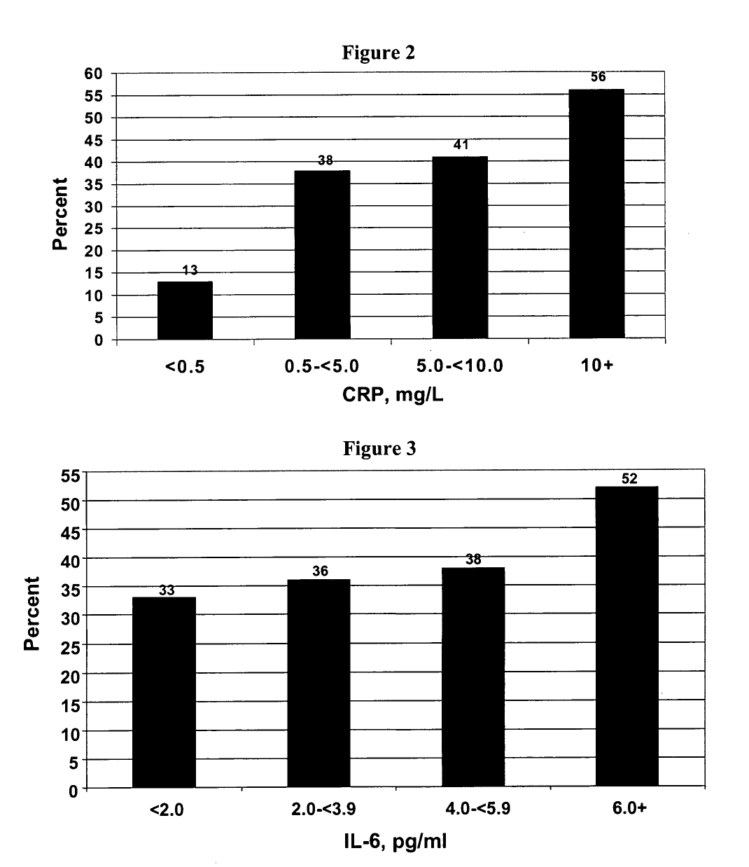

After reviewing the crude relationship between CRP, IL-6, and progression to advanced AMD, it appeared there were cutpoints (thresholds) that defined low, middle range, and high risk groups. As seen in FIGS. 2 and 3, CRP levels less than 0.5 mg/L conferred the lowest risk, and within the range of CRP from 0.5-<10.0, there was little variation in risk of AMD, whereas levels 10 or higher had the highest risk of progression of AMD. IL-6 levels 6.0 pg/ml or higher were associated with increased risk for progression of AMD.

TABLE 9
Relative Risks for Progression to Advanced AMD by Quartiles of biomarkers.

| | QUARTILE | | | | |
|---|---|---|---|---|---|
| BIOMARKER | 1 | 2 | 3 | 4 | P (Trend) |
| CRP | | | | | |
| N | 62 | 61 | 65 | 63 | |
| Progression Rate (%) | 24 | 41 | 37 | 51 | |
| Median, mg/L | 0.5 | 1.6 | 4.0 | 11.4 | |
| Range | 0.08-1.2 | 1.2-2.95 | 1.8-5.8 | 5.8-47.9 | |
| Adjusted RR | 1.0 | 1.92 | 1.74 | 2.68 | 0.005 |
| Multivariate RR (1) | 1.0 | 1.59 | 1.60 | 2.10 | 0.046 |
| (95% CI) | | (0.89-3.14) | (0.80-3.17) | (1.06-4.18) | |
| Multivariate RR (2) | 1.0 | 1.47 | 1.49 | 2.02 | 0.057 |
| (95% CI) | | (0.73-2.94)) | (0.75-2.99) | (1.00-4.04) | |
| IL-6 | | | | | |
| N | 61 | 61 | 64 | 63 | |
| Progression Rate (%) | 31 | 38 | 34 | 51 | |
| Median, pg/ml | 1.25 | 2.14 | 3.4 | 7.4 | |
| Range | 0.43-1.7 | 1.7-2.7 | 2.7-4.7 | 4.8-30 | |
| Adjusted RR | 1.0 | 1.19 | 1.4 | 2.25 | 0.005 |
| Multivariate RR (1) | 1.0 | 0.93 | 1.31 | 1.81 | 0.026 |
| (95% CI) | | (0.49-1.77) | (0.69-2.49) | (0.97-3.36) | |
| Multivariate RR (2) | 1.0 | 1.00 | 1.40 | 1.96 | 0.017 |
| (95% CI) | | (0.52-1.93) | (0.73-2.70) | (1.04-3.71) | |
| TNF - Alpha Receptor II | | | | | |
| N | 60 | 62 | 64 | 63 | |
| Progression Rate (%) | 28 | 35.5 | 44 | 46 | |
| Median, pg/ml | 2140.8 | 2918.20 | 3589.95 | 4725.90 | |
| Range | 1434-2610 | 2610-3176 | 3177-4000 | 4000-8494 | |
| Adjusted RR | 1.0 | 1.29 | 1.60 | 1.68 | 0.073 |
| Multivariate RR (1) | 1.0 | 0.94 | 1.43 | 1.3 | 0.297 |
| (95% CI) | | (0.48-1.84) | (0.74-2.75) | (0.65-2.46) | |
| Multivariate RR (2) | 1.0 | 0.93 | 1.35 | 1.19 | 0.426 |
| (95% CI) | | (0.47-1.84) | (0.70-2.62) | (0.60-2.32) | |
| VCAM | | | | | |
| N | 60 | 63 | 63 | 63 | |
| Progression Rate (%) | 27 | 43 | 38 | 46 | |
| Median, ng/ml | 717.45 | 896.3 | 1037.1 | 1318.6 | |
| Range | 441-839 | 839-977 | 978-1136 | 1137-2637 | |
| Adjusted RR | 1.0 | 1.66 | 1.5 | 2 | 0.054 |
| Multivariate RR (1) | 1.0 | 1.45 | 1.35 | 1.94 | 0.083 |
| (95% CI) | | (0.76-2.78) | (0.69-2.65) | (0.99-3.8) | |
| Multivariate RR (2) | 1.0 | 1.36 | 1.30 | 1.76 | 0.138 |
| (95% CI) | | (0.70-2.63) | (0.66-2.55) | (0.89-3.47) | |

### Example 5

### Relationship Between Biomarkers and AMD Risk Factors

Table 10 displays the results of linear regression analyses of log CRP and log IL-6 on all covariates in Tables 6-8, including known risk factors for AMD. Log values shown are after controlling for age, sex, education, initial worst eye and protein intake and variables in the table. Physical activity refers to mean number of times/per week of vigorous activity. Systolic blood pressure and log calories were continuous, e.g., actual results were used. Beta-carotene, zinc, vitamin C and vitamin E (energy adjusted) were all continuous as well.

These results demonstrate that both CRP and IL-6, markers of systemic inflammation, were significantly and independently related to AMD after adjustment for known and potential confounding factors. The highest level of CRP was significantly associated with progression of AMD with a two-fold greater risk compared with the lowest level of CRP. Similarly, the highest quartile of IL-6 was significantly related to progression of AMD with almost a two-fold risk compared with the lowest quartile. For CRP, the estimates of risk were increased above the first quartile, and the trend was marginally significant. IL-6 values in the third and forth quartiles were associated with increased risk, and the trend for increasing risk of AMD with increasing levels of IL-6 was statistically significant.

Smoking and body mass index are known risk factors associated with AMD, and current smoking as well as intermediate to high levels of body mass index had a significant, positive association with both CRP and IL-6. Thus, these two variables meet the definition of a confounder. However, even after adjustment for these and other factors, CRP and IL-6 were related to progression of AMD. Although some unmeasured and therefore uncontrolled factors might still be confounding these relationships, they would have to be both highly associated with CRP and IL-6, and a strong risk factor for AMD to substantially alter the relationships seen.

TABLE 10
Multiple Regressions of Log CRP and IL-6 on Various Covariates

| | Log CRP (N = 251) | | Log IL-6 (N = 249) | |
|---|---|---|---|---|
| Coefficient | Beta ± SE | P Value | Beta ± SE | P Value |
| Physical activity | −0.04 ± 0.03 | 0.29 | −0.03 ± 0.02 | 0.14 |
| Current smoker | 1.05 ± 0.31 | <0.001 | 0.56 ± 0.20 | 0.006 |
| Past smoker | 0.17 ± 0.18 | 0.33 | 0.18 ± 0.12 | 0.12 |
| Systolic BP, mm Hg | 0.008 ± 0.004 | 0.061 | 0.004 ± 0.003 | 0.14 |
| Cardiovascular disease | 0.33 ± 0.20 | 0.096 | 0.20 ± 0.13 | 0.11 |
| Body mass index, kg/m² (25-29) | 0.77 ± 0.19 | <0.001 | 0.36 ± 0.12 | 0.003 |
| Body mass index, kg/m² (30+) | 0.62 ± 0.22 | 0.005 | 0.32 ± 0.14 | 0.022 |
| Alcohol, g/d | −0.001 ± 0.006 | 0.91 | 0.001 ± 0.004 | 0.90 |
| Calories, (ln) | −0.08 ± 0.36 | 0.83 | 0.04 ± 0.23 | 0.85 |
| Beta-carotene intake, ug/d | −0.01 ± 0.11 | 0.92 | −0.001 ± 0.070 | 0.99 |
| Zinc, mg/d | 0.14 ± 0.11 | 0.20 | −0.02 ± 0.07 | 0.73 |
| Vitamin C, mg/d | −0.24 ± 0.011 | 0.025 | 0.05 ± 0.07 | 0.45 |
| Vitamin E, mg/d | −0.001 ± 0.055 | 0.99 | −0.06 ± 0.04 | 0.11 |

These data demonstrate that there is a relationship between certain inflammatory markers and development of more advanced AMD, in particular CRP and IL-6, and to some extent V-CAM, and that there is a slightly increased risk with the lipid biomarker ApoB. Thus, these makers can be used to determine a subject's risk of progressing to advanced AMD.

### Example 6

### Effect Modification by Intake of Linoleic Acid

In this study, the effect of levels of intake of linoleic acid was evaluated in the population described in Example 1.

Table 11 illustrates a modification of the effect of biomarkers depending on the level of linoleic acid intake, which is an omega-6 fatty acid. The “Adjusted RR” is adjusted for age-sex group (60-69 Male/70-79 Male/80+Male/60-69 Female/70-79 Female/80+Female), log calories (continuous), and protein intake (quartile). The Multivariate RR (model 1) is adjusted for age-sex group (60-69 Male/70-79 Male/80+Male/60-69 Female/70-79 Female/80+Female), education (≧high school vs. <high school), smoking (current/past/never), BMI (<25/25-29.9/30+), systolic blood pressure, cardiovascular disease, log calories (continuous), protein intake (quartile), calorie-adjusted beta carotene intake (continuous), alcohol intake (continuous), physical activity (continuous-times/wk vigorous), and initial AMD grade (categorical). The Multivariate RR (model 2) is adjusted for the variables in model 1 plus total intake of zinc, vitamin C and vitamin E. RR's (or p-values) in boldface are significant at p=0.05 level.

Higher levels of linoleic acid in combination with elevated levels of CRP are associated with an even greater risk of developing AMD than for elevated levels of CRP alone (relative risks are 3.80 and 2.30 in different statistical models). Previous reports on dietary fat and AMD show that omega-3 fatty acids (e.g., in foods and in fish) have a protective effect only when linoleic acid intake levels are below the median, e.g., in the lower two quartiles (Seddon et al., Arch Ophthalmol 2001; 119:1191-1199; Seddon et al., Arch Ophthalmol. 2003; 121:1728-1737).

TABLE 11
Relative Risks for Progression to Advanced AMD by Quartile
of CRP Within Strata of Linoleic Acid Intake

| | QUARTILE OF CRP | | | | |
|---|---|---|---|---|---|
| | 1 | 2 | 3 | 4 | P trend |
| Linoleic Acid Intake, | | | | | |
| Quartiles 1 and 2 | | | | | |
| (≦4.9 g) N = 125 | | | | | |
| Adjusted RR | 1.0 | 3.55 | 1.87 | 3.35 | .076 |
| Multivariate RR (1) | 1.0 | 1.70 | 1.01 | 1.52 | 0.81 |
| Multivariate RR (2) | 1.0 | 1.57 | 1.09 | 1.51 | 0.72 |
| Linoleic Acid Intake, | | | | | |
| Quartiles 3 and 4 | | | | | |
| (>4.9 g) N = 126 | | | | | |
| Adjusted RR | 1.0 | 1.31 | 1.745 | 1.82 | 0.039 |
| Multivariate RR (1) | 1.0 | 2.17 | 3.08 | 3.80 | 0.0013 |
| Multivariate RR (2) | 1.0 | 1.65 | 1.36 | 2.30 | 0.03 |

These data demonstrate that the adverse effect associated with high CRP levels is enhanced in the higher quartiles of linoleic acid intake. Thus, CRP levels can also be used in combination with linoleic acid intake levels to predict a subject's risk of progressing to advanced AMD. In addition, reducing the subject's levels of linoleic acid intake can be used to prevent or delay the progression to advanced AMD. Furthermore, lowering intake of linoleic acid in combination with lowering CRP levels, e.g., by administering an anti-inflammatory agent, can be used to prevent or delay progression to advanced AMD.

### Example 7

### Association Between CRP Levels and Onset of Advanced AMD

Prospective analysis of CRP levels was performed using the same population of subjects and methodology described in Example 1 (the AREDS study). An increased relative risk was seen for onset of advanced AMD (1.34 overall, 1.28 for neovascular disease, and 1.68 for geographic atrophy). Table 12 summarizes the unadjusted risk ratios for AMD events by CRP quartile.

TABLE 12
Summary of Unadjusted Risk Ratios
for AMD Events By CRP Quartile

CRP Quartile (mg/L)*

| | Q1 | Q2 | Q3 | Q4 |
|---|---|---|---|---|
| Quartile (mg/L) | 0-1.2 | 1.2-2.6 | 2.6-5.7 | 5.7-max |

Neovascular Event

| | | | | |
|---|---|---|---|---|
| N | 151 | 166 | 147 | 167 |
| # Events | 17 | 22 | 10 | 24 |
| RR | 1.00 | 1.18 | 0.60 | 1.28 |

CGA Event

| | | | | |
|---|---|---|---|---|
| N | 151 | 166 | 147 | 167 |
| # Events | 7 | 6 | 13 | 13 |
| RR | 1.00 | 0.78 | 1.91 | 1.68 |

Any Event

| | | | | |
|---|---|---|---|---|
| N | 151 | 166 | 147 | 167 |
| # Events | 23 | 27 | 22 | 34 |
| RR | 1.00 | 1.07 | 0.98 | 1.34 |

These data demonstrate that assays of CRP levels can be used to determine a subject's risk of progressing to advanced AMD.

### Example 8

### Association Between Maculopathy and Inflammatory and Lipid Biomarkers

The case control study described in this example evaluated the odds of developing AMD in the AREDS population as described in Example 1. The results, shown in Table 13, demonstrate that there is an increased risk of AMD for a number of inflammatory biomarkers, including V-CAM (OR 1.6, Q4 vs. Q1), TNF-alpha receptor II (OR 1.8, Q4 vs. Q1), and possibly IL-6 (OR 1.78, mean±2 standard deviations cut-off).

OR's (or p-values) in boldface are significant at p=0.05 level. Multivariate results are adjusted for all of the variables described in Example 1, above, plus thyroid hormones.

TABLE 13
Association between Maculopathy and
Inflammatory and Lipid Biomarkers

| | | | | | | |
|---|---|---|---|---|---|---|
| Biomarker | LP(a) | Apo B | ICAM | VCAM | IL-6 | TNF-R2 |

Age & sex adj.

| | | | | | | |
|---|---|---|---|---|---|---|
| OR (Q2 vs. Q1) | 1.43 | 1.16 | 1.15 | 1.32 | 1.2 | 1.2 |
| OR (Q3 vs. Q1) | 1.14 | 0.84 | 1.21 | 1.35 | 1.14 | 1.2 |
| OR (Q4 vs. Q1) | 1.17 | 1.16 | 1.45 | 1.6 | 1.42 | 1.82 |
| p - trend | 0.73 | 0.76 | 0.053 | 0.038 | 0.11 | 0.006 |

Multivariate*

| | | | | | | |
|---|---|---|---|---|---|---|
| OR (Q2 vs. Q1) | 1.55 | 1.21 | 1.08 | 1.26 | 1.23 | 1.19 |
| OR (Q3 vs. Q1) | 1.24 | 0.84 | 1.15 | 1.22 | 1.17 | 1.14 |
| OR (Q4 vs. Q1) | 1.27 | 1.11 | 1.29 | 1.49 | 1.49 | 1.8 |
| p - trend | 0.46 | 0.996 | 0.19 | 0.11 | 0.09 | 0.012 |

Multivariate* + CRP

| | | | | | | |
|---|---|---|---|---|---|---|
| OR (Q2 vs. Q1) | 1.47 | 1.19 | 1.1 | 1.26 | 1.15 | 1.2 |
| OR (Q3 vs. Q1) | 1.29 | 0.85 | 1.13 | 1.17 | 0.99 | 1.1 |
| OR (Q4 vs. Q1) | 1.21 | 1.11 | 1.21 | 1.46 | 1.17 | 1.72 |
| p - trend | 0.5 | 0.98 | 0.36 | 0.15 | 0.66 | 0.034 |

Multivariate* + CRP - Category 4 vs. 1

| | | | | | | |
|---|---|---|---|---|---|---|
| OR (Q2 vs. Q1) | 1.29 | 1.2 | 1.23 | 1.61 | 1.77 | 1.45 |
| OR (Q3 vs. Q1) | 1.12 | 0.75 | 1.07 | 1.81 | 1.14 | 1.53 |
| OR (Q4 vs. Q1) | 0.67 | 1.18 | 1.34 | 2.16 | 1.93 | 2.06 |

**90th percentile as cutoff**

| | | | | | | |
|---|---|---|---|---|---|---|
| Age & sex adj. | 1.04 | 1.06 | 1.2 | 1.7 | 1.14 | 1.51 |
| Multivariate* | 1.03 | 1.04 | 1.14 | 1.72 | 1.12 | 1.49 |
| Multivariate* + | 0.99 | 1.03 | 1.03 | 1.58 | 0.91 | 1.37 |
| CRP | | | | | | |

**Mean + 2 s.d. as cutoff**

| | | | | | | |
|---|---|---|---|---|---|---|
| Age & sex adj. | 1.14 | 1.72 | 1.19 | 1.3 | 1.5 | 1.61 |
| Multivariate* | 1.06 | 1.76 | 1.14 | 1.21 | 1.78 | 1.74 |
| Multivariate* + | 1.03 | 1.63 | 0.99 | 1.1 | 1.36 | 1.34 |
| CRP | | | | | | |

**Ever/never smoked (es, ns) × Tertiles (t1, t2, t3)

| | | | | | | |
|---|---|---|---|---|---|---|
| Multivariate* | 1.85 | 1.35 | 1.19 | 1.97 | 1.24 | 2.19 |
| OR (t1, es × t1, ns) | 1.6 | 1.04 | 1.21 | 1.6 | 1.09 | 1.53 |
| OR (t2, ns × t1, ns) | 2.13 | 1.47 | 1.62 | 2.16 | 1.68 | 1.91 |
| t2, es × t1, ns | 1.21 | 0.97 | 1.1 | 2.06 | 1.25 | 2.47 |
| t3, ns × t1, ns | 1.43 | 1.52 | 1.94 | 2.37 | 1.76 | 2.44 |
| t3, es × t1, ns | 1.85 | 1.35 | 1.19 | 1.97 | 1.24 | 2.19 |

Multivariate + CRP

| | | | | | | |
|---|---|---|---|---|---|---|
| t1, es × t1, ns | 1.76 | 1.33 | 1.24 | 1.87 | 1.28 | 2.24 |
| t2, ns × t1, ns | 1.56 | 1.05 | 1.27 | 1.52 | 0.99 | 1.52 |
| t2, es × t1, ns | 2.13 | 1.44 | 1.65 | 2.13 | 1.56 | 1.9 |
| t3, ns × t1, ns | 1.18 | 0.97 | 1.07 | 1.93 | 1.02 | 2.43 |
| t3, es × t1, ns | 1.39 | 1.53 | 1.88 | 2.16 | 1.41 | 2.34 |

Ever vs. Never smoked within each tertile (t1, t2, t3)
t1

| | | | | | | |
|---|---|---|---|---|---|---|
| Multivariate | 2.08 | 1.25 | 1.18 | 2.14 | 1.17 | 2.28 |
| Multivariate + CRP | 1.96 | 1.23 | 1.27 | 2.12 | 1.22 | 2.38 |

t2

| | | | | | | |
|---|---|---|---|---|---|---|
| Multivariate | 1.22 | 1.41 | 1.3 | 1.2 | 1.63 | 1.22 |
| Multivariate + CRP | 1.25 | 1.29 | 1.21 | 1.21 | 1.63 | 1.23 |

t3

| | | | | | | |
|---|---|---|---|---|---|---|
| Multivariate | 1.16 | 1.61 | 1.9 | 1.14 | 1.4 | 1.01 |
| Multivariate + CRP | 1.17 | 1.66 | 1.87 | 1.1 | 1.35 | 1.02 |

These data demonstrate that there is a relationship between inflammatory markers and AMD, in particular CRP, TNF-R2, IL-6, and V-CAM, and that there is a slightly increased risk with the lipid biomarker lipoprotein (a) (Lp(a)). Thus, these markers can be used to determine a subject's risk of developing AMD.

### Example 9

### Plasma Homocysteine and Risk of Age-Related Macular Degeneration

Homocysteine (HCY) is a sulphydryl-containing amino acid derived from the demethylation of methionine, which is found mainly in animal protein. Total plasma homocysteine level can be influenced by genetic defects, renal impairment, and various drugs and diseases. Dietary factors have also been shown to play an important role in the control of homocysteine levels, as homocysteine metabolism is dependent on reactions involving vitamins B-6, B-12, and folate for transsulfuration and remethylation. High levels of plasma homocysteine are toxic to the vascular endothelium, releasing free radicals, thus affecting the vessel walls and increasing the risk for thrombosis.

One recent study suggested a possible involvement of increased plasma homocysteine levels in AMD (Axer-Siegel et al., Am J Ophthalmol 2004; 137: 84-89), but another did not (Heuberger et al., Am J Clin Nutr 2002; 76: 897-902). However, these studies had few cases of AMD and one did not evaluate advanced stages of the disease. To examine the correlation between homocysteine levels and AMD, we studied the relationship between levels of fasting plasma homocysteine levels and AMD in large well-characterized population at two centers in the US. The odds ratio (ORs) and confidence interval (CI) results are shown in Table 14.

TABLE 14
Summary of ORs and CI's for HCY

| Adjustment | Comparison | OR | CI |
|---|---|---|---|
| Age & Gender | | | |
| | Q2 vs Q1 | 0.79 | (0.54, 1.17) |
| | Q3 vs Q1 | 0.97 | (0.66, 1.41) |
| | Q4 vs Q1 | 1.11 | (0.74, 1.66) |
| | (p-for trend for above = .44) | | |
| | Continuous: 5-unit change | 1.12 | (0.90, 1.39) |
| | Above/Below 90% tile | 1.43 | (0.92, 2.21) |
| | >95% vs. <90% tile | 1.39 | (0.79, 2.46) |
| | Above/Below mean + 2 s.d. | 1.39 | (0.72, 2.67) |
| | Above/below HCY = 12 | 1.62 | (1.09, 2.41) |
| Multiv. | | | |
| | Q2 vs Q1 | 0.80 | (0.54, 1.20) |
| | Q3 vs Q1 | 0.99 | (0.67, 1.46) |
| | Q4 vs Q1 | 1.08 | (0.71, 1.64) |
| | (p-for trend for above = .52) | | |
| | Continuous: 5-unit change | 1.12 | (0.90, 1.40) |
| | Above/Below 90% tile | 1.39 | (0.89, 2.18) |
| | >95% vs. <90% tile | 1.33 | (0.74, 2.37) |
| | Above/Below mean + 2 s.d. | 1.31 | (0.67, 2.54) |
| | Above/below HCY = 12 | 1.60 | (1.07, 2.41) |
| Multiv + CRP | | | |
| | Q2 vs Q1 | 0.79 | (0.53, 1.19) |
| | Q3 vs Q1 | 0.94 | (0.63, 1.40) |
| | Q4 vs Q1 | 1.04 | (0.68, 1.60) |
| | (p-for trend for above = .67) | | |
| | Continuous: 5-unit change | 1.10 | (0.87, 1.38) |
| | Above/Below 90% tile | 1.36 | (0.86, 2.16) |
| | >95% vs. <90% tile | 1.28 | (0.71, 2.30) |
| | Above/Below mean + 2 s.d. | 1.26 | (0.64, 2.47) |
| | Above/below HCY = 12 | 1.58 | (1.05, 2.39) |

Q1 was the First Quartile (3.88-7.43 mmol/L); Q2, the Second Quartile (7.43-8.81 nmol/L); Q3, the Third Quartile (8.81-11.07 mol/L); and Q4, the Fourth Quartile (11.07-33.0 nmol/L). The 90^{th}% tile was 12.82 nmol/L, and the 95^{th}% tile was 14.59 nmol/L. The mean+2 SD (standard deviations) was 14.94 nmol/L.

Multivariate adjustment corrected for age, sex, race, smoking, education, BMI, dietary antioxidant intake, diabetes, history of CVD, hypertension, and taking AREDS antioxidants (randomly assigned).

Median plasma levels of HCY were statistically significantly higher among subjects who had more advanced maculopathy. The median value for group 4 was 9.51 nmol/L and the median value for group 1 was 8.81 mmol/L (p=0.01). Since homocysteine levels above 12 nmol/L are considered abnormal, this relationship was also evaluated; homocysteine levels above 12 nmol/L were associated with higher risk of AMD, controlling for other factors (OR 1.6), and controlling for CRP (1.58) and these associations were statistically significant (multivariate p=0.02). In various multivariate adjusted models, there were small, positive associations between increased HCY and increased risk of intermediate or advanced AMD when HCY was evaluated categorically or continuously (Table 14), but these were not significant. Persons above the 90th percentile of HCY were more likely to have intermediate or advanced AMD (OR=1.39; 95% CI, 0.89-2.18), but this result was not statistically significant. It was explored whether an effect of HCY would be modified by smoking (never, ever), but there was no consistent association seen, and persons in the highest level of HCY had non-significant higher risk of AMD, (OR 1.38), regardless of smoking category.

These results are consistent with a possible small to moderate association between high HCY levels and AMD.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for determining a subject's risk of development or progression of age-related macular degeneration (AMD), the method comprising
obtaining a level of an AMD biomarker in the subject; and
comparing the level of the biomarker to a reference,
wherein the subject's risk of development or progression of AMD is based upon the level of the biomarker in comparison to the reference.

2. The method of claim 1, wherein the progression of AMD is progression to advanced AMD.

3. The method of claim 1, wherein the biomarker is a marker of systemic inflammation or a lipid biomarker.

4. The method of claim 3, wherein the AMD biomarker is selected from the group consisting of C-reactive protein (CRP), interleukin-6 (IL-6), Apolipoprotein B (ApoB), TNF-alpha receptor II (TNF-R2), homocysteine (HCY), and Vascular Cell Adhesion Molecule (VCAM).

5. The method of claim 3, wherein the marker of systemic inflammation is C-reactive protein (CRP),

6. The method of claim 3, wherein the marker of systemic inflammation is interleukin-6 (IL-6).

7. The method of claim 1, wherein the reference is a single predetermined value, if the level of the biomarker in the subject is higher than the predetermined value, then the subject has an increased risk, and if the level of the biomarker in the subject is higher than predetermined value, then the subject has a reduced risk.

8. The method of claim 7, wherein the predetermined value is selected from the group consisting of the values listed in Tables A and B.

9. The method of claim 1, wherein the reference is a plurality of biomarker level ranges defining lowest, average and highest risk categories, and the comparing step comprises determining into which of the biomarker level ranges the subject's level falls.

10. The method of claim 9, wherein one or more of the plurality of biomarker level ranges is selected from the group consisting of the ranges listed in Tables A or B.

11. The method of claim 1, wherein the subject is apparently healthy.

12. The method of claim 7, wherein the subject's risk is increased, and the method further comprises administering to the subject a therapeutically effective amount of an anti-inflammatory agent.

13. The method of claim 12, wherein the anti-inflammatory agent is selected from the group consisting of statins and aspirin.

14. The method of claim 9, wherein the subject's level falls in the average or highest risk category, and the method further comprises administering to the subject a therapeutically effective amount of an anti-inflammatory agent.

15. The method of claim 14, wherein the anti-inflammatory agent is selected from the group consisting of statins and aspirin.

16. The method of claim 1, further comprising:
administering to the subject at least one dose of an anti-inflammatory agent;
obtaining a second level of the AMD biomarker in the subject, and
comparing the second level of the AMD biomarker to a reference,
wherein the subject's risk of development or progression of AMD after administration of the anti-inflammatory agent is based upon the second level of the biomarker in comparison to the reference.

17. The method of claim 16, wherein the reference is a level of an AMD biomarker in the subject obtained prior to administration of the anti-inflammatory agent.

18. The method of claim 1, further comprising
obtaining a level of a second AMD biomarker in the subject, and
comparing the level of the second biomarker to a second reference,
wherein the subject's risk of developing AMD is based upon the level of the second biomarker in comparison to the second reference.

19. The method of claim 18, wherein the first and second marker are C-reactive protein (CRP) and interleukin-6 (IL-6), respectively.

20. A method for determining a subject's risk of developing AMD or progression of AMD, the method comprising:
obtaining a level of an AMD biomarker in the subject,
comparing the level of the biomarker to a first reference to establish a first risk value,
obtaining a level of a risk factor in the subject,
comparing the level of the risk factor to a second reference to establish a second risk value, and
determining the subject's risk of developing AMD or progression of AMD based upon the first risk value and the second risk value.

21. The method of claim 21, wherein the first risk value and second risk value are used to establish a combined risk value.

22. The method of claim 22, wherein the combined risk value is greater than either of the first and second risk values, or greater than the sum of the first and second risk values.

23. The method of claim 21, wherein the biomarker is selected from the group consisting of C-reactive protein (CRP), interleukin-6 (IL-6), TNF-alpha receptor II (TNF-R2), Apolipoprotein B (ApoB), homocysteine (HCY), and Vascular Cell Adhesion Molecule (VCAM).

24. The method of claim 21, wherein the risk factor is selected from the group consisting of family history of AMD, age, sex, smoking history, obesity, body mass index (BMI), waist circumference, waist-hip ratio, weight change since age 20, dietary fat intake, linoleic acid intake, and elevated cholesterol levels.

25. A method of selecting a subject or a population of subjects for participation in a clinical trial of a treatment for age-related macular degeneration, the method comprising:
obtaining a level of an AMD biomarker in a subject, and
comparing the level of the AMD biomarker to a reference, wherein the level of the biomarker in comparison to the reference is indicative of whether the subject should participate in the clinical trial.

26. The method of claim 26, wherein the biomarker is selected from the group consisting of C-reactive protein (CRP), interleukin-6 (IL-6), Apolipoprotein B (ApoB), TNF-alpha receptor II (TNF-R2), homocysteine (HCY), and/or Vascular Cell Adhesion Molecule (VCAM).

27. A method of monitoring a treatment for reducing risk of development of age-related macular degeneration (AMD) or progression to advanced AMD, the method comprising:
obtaining a first level of an AMD biomarker in a subject;
administering a selected treatment to the subject;
obtaining a second level of the AMD biomarker in the subject; and
comparing the first level of the biomarker to the second level of the biomarker,
wherein a change or no change in the second level as compared to the first level indicates whether the treatment is effective or not effective.

28. The method of claim 27, wherein the second level is lower than the first level, indicating that the treatment or prevention is effective.

29. The method of claim 27, wherein the second level is higher than or the same as the first level, indicating that the treatment or prevention is not effective or not yet effective.

30. The method of claim 28, wherein the AMD biomarker is selected from the group consisting of C-reactive protein (CRP), interleukin-6 (IL-6), Apolipoprotein B (ApoB), TNF-alpha receptor II (TNF-R2), homocysteine (HCY), and Vascular Cell Adhesion Molecule (VCAM).

31. A kit for determining a subject's risk of development or progression of age-related macular degeneration (AMD), the kit comprising one or more assays for an AMD biomarker and a reference, wherein the assay results provide a level of the AMD biomarker for determining the subject's risk of development or progression of AMD by comparing the level of the AMD biomarker determined by the assay with a reference to determine the subject's risk of development or progression of AMD.

32. The kit of claim 31, wherein the reference is a single predetermined value or a plurality of biomarker level ranges.

33. The kit of claim 32, wherein the single predetermined value or a plurality of biomarker level ranges is selected from the group consisting of the values listed in Tables A and B.
